(19) United States
(12) Patent Application Publication
(10) Pub. No.: US 2015/0166642 A1
(43) Pub. Date: Jun. 18, 2015

(54) METHODS AND COMPOSITIONS FOR PEROXISOME PROLIFERATOR-ACTIVATED RECEPTOR GAMMA COACTIVATOR-1alpha (PGC1alpha) AS A TARGET OF CIRCULATING TUMOR CELLS

(71) Applicant: BETH ISREAL DEACONESS MEDICAL CENTER, INC., Boston, MA (US)

(72) Inventors: Raghu Kalluri, Weston, MA (US); Valerie S. Lebleu, Brookline, MA (US)

(21) Appl. No.: 14/400,687

(22) PCT Filed: May 15, 2013
(86) PCT No.: PCT/US2013/041106

§ 371 Date: Nov. 12, 2014

Related U.S. Application Data
(60) Provisional application No. 61/647,172, filed on May 15, 2012.

(51) Int. Cl. C07K 16/18; A61K 31/713; C12N 15/113; G01N 33/574; C12Q 1/68; A61K 39/395; A61K 45/06

(52) U.S. Cl. CPC C07K 16/18; A61K 39/3955; A61K 31/713; A61K 45/06; G01N 33/57496; C12Q 1/6886; C12N 15/113; C07K 2317/76; G01N 2333/4706; C12Q 2600/158; C12Q 2600/16; C12N 2310/14

(57) ABSTRACT

The present invention relates to methods, compositions, and diagnostic tests for treating and diagnosing a metastatic as disease that results in increased mitochondrial respiration and/or biogenesis. In particular, the methods and compositions include treatment of metastatic diseases such as breast cancer using an antagonist of mitochondrial respiration such as a PGC1α antagonist.

### CROSS-REFERENCE TO RELATED APPLICATIONS

This application claims benefit of priority to U.S. Provisional Application No. 61/647,172, filed May 15, 2012 which is hereby incorporated by reference.

### BACKGROUND OF THE INVENTION

The glucose metabolism diversion of cancer cells to promote rapid ATP production per unit time, via high glycolytic rate and lactate production rather than oxidative phosphorylation, is believed to adequately meet the energy expenditure of rapidly proliferating cancer cells by supporting the anabolic accumulation of biosynthetic precursors. It is, however, becoming clear that despite enhanced glycolysis, cancer cells also operate mitochondrial respiration to derive a significant fraction of their ATP. The initial autonomous metabolic reprogramming of rapidly proliferating cancer cells promotes self-sustaining signal transduction mechanisms to foster growth regulatory properties in those cells. In the growing tumor, this adaptive metabolic reprogramming, precipitated in part by oncogenic transformation, not only gives cancer cells a proliferative advantage but likely engages the tumor stroma to further enrich the growth advantageous milieu of rapidly proliferating cells. Nevertheless, the metabolic requirement of invasive and metastatic cancer cells that suspend their proliferative program to acquire a migratory phenotype remains unknown. Whether the metabolic profile of invasive and circulating tumor cells differs from the metabolic profile of proliferative cancer cells in the primary tumor is undetermined.

### SUMMARY OF THE INVENTION

The invention features a method of treating a subject having a metastatic disease, the method including administering to the subject an antagonist of mitochondrial respiration, in an amount sufficient to treat the metastatic disease.

The invention also features a method of treating a subject having a metastatic disease, the method including determining the level of mitochondrial respiration in a sample from the subject and administering to a subject having increased levels of mitochondrial respiration an antagonist that inhibits mitochondrial respiration in an amount sufficient to treat the metastatic disease.

In one aspect, the level of mitochondrial respiration is determined based on increased PGC1α activity.

In some embodiments, the sample includes cancer cells. In particular embodiments, the cancer cells are circulating tumor cells.

The invention also features a method for diagnosing a subject as having, or having a predisposition to a metastatic disease, the method including, determining the level of mitochondrial respiration in a sample from the subject, comparing the level of mitochondrial respiration with a normal reference sample, wherein the presence of an increased level of mitochondrial respiration, as compared to the normal reference sample, results in diagnosing the subject as having, or having a predisposition to the metastatic disease and, administering to the subject an antagonist that inhibits mitochondrial respiration, in an amount sufficient to treat the metastatic disease.

For any of the methods or compositions described herein, the antagonist is an RNAi agent, a small molecule inhibitor, or an antibody.

In some embodiments, the small molecule inhibitor can be selected from the group consisting of: atractyloside, bongkrekic acid, carbonyl cyanide m-chlorophenylhydrazone, carboxyatractyloside, CGP-37157, erastin, F16, hexokinase II inhibitor II, 3-BP, and (−)-deguelin.

In some embodiments, the antagonist is a PGC1α antagonist. In other embodiments, the PGC1α antagonist is an RNAi agent, or an anti-PGC1α antibody.

In any of the embodiments described herein, the antagonist can be administered with an anticancer agent.

In any of the embodiments described herein, the metastatic disease can be selected from the group consisting of: leukemia, brain cancer, bladder cancer, breast cancer, cervical cancer, colorectal cancer, endometrial cancer, esophageal cancer, head and neck cancer, liver cancer, lung cancer, lymphoma, ovarian cancer, pancreatic cancer, prostate cancer, renal cancer, skin cancer, stomach cancer, testis cancer, thyroid cancer, and urothelial cancer.

In particular embodiments, the metastatic disease is breast cancer, In another embodiment, the breast cancer is selected from the group consisting of: ductal carcinoma, invasive ductal carcinoma, tubular carcinoma, medullary carcinoma, mucinous carcinoma, papillary carcinoma, cribriform carcinoma, invasive lobular carcinoma, inflammatory breast cancer, lobular carcinoma, male breast cancer, Paget's Disease, and phyllodes tumors.

### DEFINITIONS

By “amount sufficient” of an agent is meant the amount of the agent sufficient to effect beneficial or desired result (e.g., treatment of a metastatic disease, e.g., breast cancer), and, as such, an amount sufficient of the formulation is an amount sufficient to achieve a reduction in the expression level and/or activity of the PGC1α gene or protein, or mitochondrial respiration/biogenesis, as compared to the response obtained without administration of the composition.

By “antagonist of mitochondrial respiration” is meant an agent or compound that decreases or reduces gene expression, protein expression, or activity (e.g., enzymatic activity) of a protein involved in and/or associated with mitochondrial respiration/biogenesis (e.g., PGC1α/β, p38, NADH dehydrogenase, succinate dehydrogenase, cytochrome bc₁ complex, cytochrome c oxidase, citrate synthease, aconitase, isocitrate dehydrogenase, succinyl-CoA synthetase, succinic dehydrogenase, fumarase, malate dehydrogenase, α-ketoglutarate dehydrogenase, malate dehydrogenase, pyruvate carboxylase, pyruvate dehydrogenase, acyl-CoA dehydrogenase, enoyl-CoA hydratase, and 3-hydroxyacyl-CoA dehygrogenase), compared to a control (e.g., a decrease by 5%, 10%, 15%, 20%, 25%, 30%, 35%, 40%, 45%, 50%, 55%, 60%, 65%, 70%, 75%, 80%, 85%, 90%, or 95%, as compared to a control or a normal reference sample), as defined herein. Antagonists of mitochondrial respiration can be identified and tested by any useful method known in the art.

By “increased mitochondrial respiration” is meant an increase in gene expression, protein expression, or activity (e.g., enzymatic activity) of any proteins involved in and/or associated with mitochondrial respiration (e.g., PGC1α/β, p38, NADH dehydrogenase, succinate dehydrogenase, cytochrome bc₁ complex, cytochrome c oxidase, citrate synthease, aconitase, isocitrate dehydrogenase, succinyl-CoA synthetase, succinic dehydrogenase, fumarase, malate dehydrogenase, α-ketoglutarate dehydrogenase, malate dehydrogenase, pyruvate carboxylase, pyruvate dehydrogenase, acyl-CoA dehydrogenase, enoyl-CoA hydratase, and 3-hydroxyacyl-CoA dehygrogenase), as compared to a control from a normal cell or normal tissue (e.g., an increase of at least 2-fold, e.g., from about 2-fold to about 150-fold, e.g., from 5-fold to 150-fold, from 5-fold to 100-fold, from 10-fold to 150-fold, from 10-fold to 100-fold, from 50-fold to 150-fold, from 50-fold to 100-fold, from 75-fold to 150-fold, or from 75-fold to 100-fold, as compared to a control or a normal reference sample). An increase in mitochondrial respiration can be determined using any useful methods known in the art. For example, an increase in mitochondrial respiration can be determined as an increase in gene expression or increase in protein concentration (e.g., as determined by PCR or gel electrophoresis) of a protein involved in an/or associated with mitochondrial respiration, as compared to a control (e.g., a sample including normal cell or normal tissue from one or more healthy subjects) or a normal reference sample, as defined herein. In another example, an increase in mitochondrial respiration can be determined directly by measuring the increase in enzymatic activity of proteins involved in and/or associated with mitochondrial respiration, and/or indirectly by measuring increase in metabolite formation (e.g., NADPH formation, NADP+/NADPH ratio, ATP formation, ATP/ADP ratio, citrate, cis-aconitate, D-isocitrate, α-ketoglutarate, succinyl-CoA succinate, fumarate, malate, oxaloacetate, and acetyl-CoA, pyruvate, e.g., from 2-fold to 4-fold, e.g., about 3-fold, increased levels, e.g. from 50-fold to 150-fold, e.g., from 75-fold to 150-fold, e.g., about 90-fold, increased levels), as compared to a control or a normal reference sample.

By “reference sample” is meant any sample, standard, standard curve, or level that is used for comparison purposes. A “normal reference sample” can be, for example, a prior sample taken from the same subject; a sample from a normal healthy subject; a sample from a subject not having a disease associated with increased mitochondrial respiration (e.g., a metastatic disease, e.g., breast cancer); a sample from a subject that is diagnosed with a propensity to develop a disease associated with increased mitochondrial respiration (e.g., metastatic disease, e.g., breast cancer), but does not yet show symptoms of the disorder; a sample from a subject that has been treated for a disease associated with increased mitochondrial respiration (e.g., metastatic disease, e.g., breast cancer); or a sample of purified protein involved in and/or associated with mitochondrial respiration (e.g., NADH dehydrogenase, PGC1α/β, p38, succinate dehydrogenase, cytochrome bc₁ complex, cytochrome c oxidase, citrate synthease, aconitase, isocitrate dehydrogenase, succinyl-CoA synthetase, succinic dehydrogenase, fumarase, malate dehydrogenase, α-ketoglutarate dehydrogenase, malate dehydrogenase, pyruvate carboxylase, pyruvate dehydrogenase, acyl-CoA dehydrogenase, enoyl-CoA hydratase, and 3-hydroxyacyl-CoA dehygrogenase).

By “increase level of PGC1α activity” is meant an increase in PGC1α gene expression, protein expression, or activity, as compared to a control from a normal cell or normal tissue (e.g., an increase of at least 2-fold, e.g., from about 2-fold to about 150-fold, e.g., from 5-fold to 150-fold, from 5-fold to 100-fold, from 10-fold to 150-fold, from 10-fold to 100-fold, from 50-fold to 150-fold, from 50-fold to 100-fold, from 75-fold to 150-fold, or from 75-fold to 100-fold, as compared to a control or a normal reference sample). Increased level of activity can be determined using any useful methods known in the art. For example, an increased level of activity can be determined as an increase in PGC1α gene expression or increased in PGC1α protein concentration (e.g., as determined by PCR or gel electrophoresis), as compared to a control (e.g., a sample including normal cell or normal tissue from one or more healthy subjects) or a normal reference sample, as defined herein. In another example, an increase level of activity can be determined as an increase in expression of one or more genes regulated by PGC1α (e.g., genes functioning in angiogenesis, e.g., ANGP2, and VEGF, genes involved in Ca²⁺-dependent signaling pathways, e.g., PPP3Cα, genes functioning in carbohydrate/glucose metabolism, e.g., PDK4, genes functioning in fatty acid metabolism/mitochondrial biogenesis, e.g., PGC1β, genes associated with insulin signaling, e.g., FOXO1, GLUT4, and genes functioning in mitogen-activated protein kinase signaling, e.g., MAPK14, and MEF2, e.g., from 3-fold to 4-fold, from 5-fold to 15-fold, from 50-fold to 150-fold increased expression, e.g., from 75-fold to 150-fold, e.g., about 90-fold increased expression), compared to a control or a normal reference sample.

By “RNAi agent” is meant any agent or compound that exerts a gene silencing effect by hybridizing a target nucleic acid. RNAi agents include any nucleic acid molecules that are capable of mediating sequence-specific RNAi (e.g., under stringent conditions), for example, a short interfering RNA (siRNA), double-stranded RNA (dsRNA), microRNA (miRNA), short hairpin RNA (shRNA), short interfering oligonucleotide, short interfering nucleic acid, short interfering modified oligonucleotide, short interfering nucleic acid, short interfering modified oligonucleotide, chemically-modified siRNA, post-transcriptional gene silencing RNA (ptgsRNA), and Dicer-substrate RNA (DsiRNA).

By “cancer cells” is meant cells that grow and divide at an unregulated, quickened pace.

By “circulating tumor cells” is meant cells that have detached from a primary tumor and circulate in the bloodstream. Circulating tumor cells may constitute seeds for subsequent growth of additional tumors (i.e. metastasis) in different tissues.

By “metastatic disease” is meant a condition characterized by rapidly dividing cells resulting in uncontrolled growth of new tissue, parts, and/or surrounding cells.

### BRIEF DESCRIPTION OF THE DRAWINGS

FIGS. 1A-1E show circulating tumor cells (CTC) exhibiting enhanced oxidative phosphorylation. FIG. 1A shows 4T1-GFP+ cells injected orthotopically in the breast pad of mice and breast cancer cells (BCC), circulating tumor cells (CTC) and cancer cells from lung metastases (LCC) FACS purified for gene expression profiling assay. FIG. 1B shows a Microarray heat map of differentially regulated genes and sample clustering of CTC, BCC and LCC. FIG. 1C. Gene profiling assay shows mitochondrial dysfunction and oxidative phosphorylation canonical pathways are the two most differentially regulated gene sets of CTC compared to BCC. FIG. 1D shows a Microarray heat map of differentially regulated genes in indicated metabolism pathways (*p0.05). Arrows point to the significant upregulation of genes associated with mitochondrial dysfunction and oxidative phosphorylation in CTC. FIG. 1E shows a Real-time QPCR analyses of relative expression of indicated genes in CTC and LCC normalized to BCC (t test, *p<0.05).

FIGS. 2A-2K show that increased PCG1α expression and increased mitochondrial biogenesis is associated with circulating tumor cells (CTC). FIG. 2A is a representative image of FACS purified CTC based on their GFP expression. Scale bar: 50 μm. FIG. 2B shows PGC1α expression, FIG. 2C shows the relative oxygen consumption rate (OCR). FIG. 2D shows the ATP/ADP ratio, and FIG. 2E shows the mitochondrial DNA (mtDNA) content in BCC, CTC and LCC from 4T1 orthotopic tumor model. FIG. 2F shows PGC1α expression and FIG. 2G shows the mitochondrial DNA (mtDNA) content in BCC, CTC and LCC from MMTV-PyMT tumor model. FIG. 2H shows PGC1α expression and FIG. 2I shows the mitochondrial DNA (mtDNA) content in BCC, CTC and LCC from MDA-MB-231 tumor model. FIG. 2J shows PGC1α expression and FIG. 2K shows mitochondrial DNA (mtDNA) content in SCC, CTC and LCC from B16F10 tumor model. SCC: Skin Cancer Cells. (t-test, *p<0.05). Data is represented as mean+/−SEM.

FIGS. 3A-3J show the analysis of PGC1α expression in 4T1 metastatic mouse breast adenocarcinoma cells. FIG. 3A shows the relative PGC1α expression in 4T1shPGC1α normalized to 4T1shScrb1 cells (t-test, p<0.05). FIG. 3B shows a Western blot for PGC1α in 4T1shPGC1α and 4T1shScrb1 cells and band intensity quantitation of 4T1shPGC1α normalized to 4T1shScrb1 cells (t-test, p<0.05). C. Relative mitochondrial DNA (mtDNA) content in 4T1shPGC1α normalized to 4T1shScrb1 cells (t-test, p<0.05). FIG. 3D shows the mitochondrial protein content relative to total cell protein content in 4T1shPGC1α normalized to 4T1shScrb1 cells (t-test, p<0.05). FIG. 3E shows the mitochondria count and representative bright field images (t-test, p<0.05). FIG. 3F shows oxygen consumption rate (OCR) in 4T1shPGC1α normalized to 4T1shScrb1 cells (t-test, p<0.05). FIG. 3G shows the ATP/ADP ratio in 4T1shPGC1α normalized to 4T1shScrb1 cells (t-test, p<0.05). FIG. 3H is a heat map rendering of the metabolites in the indicated metabolism pathways. FIG. 3I shows the ratio of ¹³C labeled metabolite peak intensity relative to unlabeled (¹²C) metabolite derived from labeled glucose fed to 4T1shPGC1α and 4T1shScrb1 cells and LC-MS/MS analyses. FIG. 3J shows real-time PCR analyses of relative expression of indicated genes in 4T1shPGC1α normalized to 4T1shScrb1 cells, and 4T1shPGC1α and 4T1shScrb1 cells with adenoviral over-expression of PGC1α, also normalized to 4T1shScrb1 cells. Mit.B.: mitochondria biogenesis, Ox.Phos: Oxidative phosphorylation, LB: lipid biosynthesis, EMT: epithelial to mesenchymal transition. (t-test, p<0.05). Data is represented as mean+/−SEM.

FIGS. 4A-4I show the analysis of PGC1α expression in B19F10 metastatic mouse melanoma cells. FIG. 4A shows the relative PGC1α expression in B16F10shPGC1α normalized to B16F10shScrb1 cells (t-test, p<0.05). FIG. 4B is a Western blot for PGC1α in B16F10shPGC1α and B16F10shScrb1 cells and band intensity quantitation of B16F10shPGC1α normalized to B16F10shScrb1 cells (t-test, p<0.05). FIG. 4C is the relative mitochondrial DNA (mtDNA) content in B16F10shPGC1α normalized to B16F10shScrb1 cells (t-test, p<0.05). FIG. 4D shows the mitochondrial protein content relative to total cell protein content in B16F10shPGC1α normalized to B16F10shScrb1 cells. FIG. 4E shows the mitochondria count and representative bright field images (t-test, p<0.05). FIG. 4F shows the oxygen consumption rate (OCR) in B16F10shPGC1α normalized to B16F10shScrb1 cells (t-test, p<0.05). FIG. 4G shows the ATP/ADP ratio in B16F10shPGC1α normalized to B16F10shScrb1 cells (t test, p<0.05). FIG. 4H shows the ratio of ¹³C labeled metabolite peak intensity relative to unlabeled (¹²C) metabolite derived from labeled glucose fed to B16F10shPGC1α and B16F10shScrb1 cells by LCMS/MS. FIG. 4I shows real-time PCR analyses of relative expression of indicated genes in B16F10shPGC1α normalized to B16F10shScrb1 cells, and B16F10shPGC1α and B16F10shScrb1 cells with adenoviral over-expression of PGC1α, also normalized to B16F10shScrb1 cells. Mit.B.: mitochondria biogenesis, Ox.Phos: Oxidative phosphorylation, LB: lipid biosynthesis, EMT: epithelial to mesenchymal transition. (t test, p<0.05) Data is represented as mean+/−SEM.

FIGS. 5A-5I show the analysis of PGC1α expression in MDA-MB 231 human metastatic breast adenocarcinoma cells. FIG. 5A shows the relative PGC1α expression in MDA-MB-231shPGC1α normalized to MDAMB-231shScrb1 cells (t-test, p<0.05). FIG. 5B shows a Western blot for PGC1α in MDA-MB-231shPGC1α and MDA-MB-231shScrb1 cells and band intensity quantitation of MDA-MB-231shPGC1α normalized to MDA-MB-231shScrb1 cells (t-test, p<0.05). FIG. 5C shows the relative mitochondrial DNA (mtDNA) content in MDA-MB-231shPGC1α normalized to MDA-MB-231shScrb1 cells (t test, p<0.05). FIG. 5D shows the mitochondrial protein content relative to total cell protein content in MDA-MB-231shPGC1α normalized to MDA-MB-231shScrb1 cells (t test, p<0.05). FIG. 5E shows the mitochondria count and representative bright field images (t test, p<0.05). FIG. 5F shows the oxygen consumption rate (OCR) in MDA-MB-231shPGC1α normalized to MDA-MB-231shScrb1 cells (t-test, p<0.05). G. ATP/ADP ratio in MDA-MB-231shPGC1α normalized to MDAMB-231shScrb1 cells (t-test, p<0.05). FIG. 5H shows the ratio of ¹³C labeled metabolite peak intensity relative to unlabeled (¹²C) metabolite derived from labeled glucose fed to MDA-MB-231shPGC1α and MDA-MB-231shScrb1 cells by LC-MS/MS. FIG. 5I shows real-time PCR analyses of relative expression of indicated genes in MDA-MB-231shPGC1α normalized to MDA-MB-231shScrb1 cells, and MDA-MB-231shPGC1α and MDA-MB-231 shScrb1 cells with adenovial over-expression of PGC1α, also normalized to MDA-MB-231shScrb1 cells. Mit.B.: mitochondria biogenesis, Ox.Phos: Oxidative phosphorylation, LB: lipid biosynthesis, EMT: epithelial to mesenchymal transition. (t test, p<0.05). Data is represented as mean+/−SEM.

FIGS. 6A-6G show that PGC1α expression induces an invasive phenotype of cancer cells. FIG. 6A is a migration assay of indicated cell lines, with and without hypoxia stimulation (t-test, *p<0.05). Expression levels are normalized to non-migrated cells, arbitrarily set to 1. FIG. 6B shows relative PGC1α expression in migrated cells compared to nonmigrated cells, with and without hypoxia stimulation (t-test, *p<0.05). FIG. 6C shows hematoxylin stained cells following invasion and quantitation of invasion assay (t-test, *p<0.05). FIG. 6D shows light microscopy imaging of migrated cells in scratch assay and quantitation of migration assay (t test, *p<0.05). FIG. 6E shows an average doubling time of indicated cells lines. FIG. 6F shows percent alive cells in anoikis assay (t test, ns=not significant). FIG. 6G is a Type I collagen gel contraction of indicated cells (t test, *p<0.05). OE: over-expression. Data is represented as mean+/−SEM.

FIGS. 7A-7U are results showing that loss in PGC1α expression suppresses cancer cell dissemination and metastasis. FIGS. 7A, C, E show tumor volume measured over time (t-test, ns=not significant). FIGS. 7B, D, F show tumor weight at experimental endpoint (t test, ns=not significant). FIGS. 7G, L, Q show FACS analysis of percent of GFP⁺ (cancer cells) cells per 200 μl blood collected at experimental endpoint (t-test, *p<0.05). FIGS. 7H, M, R show number of CTC colonies (t-test, *p<0.05). FIGS. 7I, N, S are representative images of H&E stained lung sections (scale bar: 0.6 mm) and magnified lung metastases (encircled in insert, scale bar: 50 μm). Arrows point to metastatic lung nodules. FIGS. 7J, O, T. Percent metastatic lung surface area relative to total lung surface area (t test, *p<0.05). FIGS. 7K, P, U show number of lung surface nodules (t-test, *p<0.05). Number of mice per group: 4T1shScrb1: n=6, 4T1shPGC1α: n=7, MDA-MB-231shScrb1: n=5, MDA-MB-231shPGC1α: n=5, B16F10shScrb1: n=5, B16F10shPGC1α: n=5). Data is represented as mean+/−SEM.

FIGS. 8A-8I show that loss in PGC1α expression suppresses cancer cells extravasation and prevents metastatic colonization. FIGS. 8A, D, G are representative images of H&E stained lung sections of mice with i.v. injection of indicated cells (scale bar: 0.6 mm) and magnified lung metastases (encircled in insert, scale bar: 50 μm). Arrows point to lung nodules. FIGS. 8B, E, H show percent metastatic lung surface area relative to total lung surface area (t-test, *p<0.05), i.v. injected cells. FIGS. 8C, F, I show the number of lung surface nodules, i.v. injected cells (t-test, *p<0.05). Number of mice per group: 4T1shScrb1: n=5, 4T1shPGC1α: n=6, MDA-MB-231shScrb1: n=5, MDA-MB-231shPGC1α: n=5, B16F10shScrb1: n=6, B16F10shPGC1α: n=5). Data is represented as mean+/−SEM.

FIGS. 9A-9H show that the functional motility of cancer cells with EMT program is dependent on PGC1α. FIG. 9A shows the relative PGC1α expression in FACS purified GFP⁺/αSMA- and GFP⁺/αSMA⁺ cells from 4T1 primary tumor (t-test, *p<0.05). FIG. 9B shows a representative CK8 (red) and αSMA (green) immunolabeling of the primary tumor. Nuclear staining (DAPI, blue). Arrows point to double positive (CK8⁺/αSMA⁺) cells. Scale bar: 100 μm. Bar graph: quantitation of number of CK8⁺/αSMA⁺ cells per field of view (t-test, ns=not significant). FIG. 9C shows the relative expression of indicated genes in 4T1shPGC1α tumors normalized to 4T1shScrb1 tumors (t-test, ns=not significant). FIG. 9D shows the relative PGC1α expression in FACS purified GFP⁺/αSMA- and GFP⁺/αSMA⁺ cells from MDA-MB-231 primary tumor (t-test, *p<0.05). FIG. 9E shows a relative expression of indicated genes in MDA-MB-231shPGC1α tumors normalized to MDA-MB-231shScrb1 tumors (t-test, ns=not significant). FIG. 9F shows a relative PGC1α expression in FACS purified GFP⁺/αSMA- and GFP⁺/αSMA⁺ cells from B16F10 primary tumor (t-test, *p<0.05). FIG. 9G shows the relative expression of indicated genes in B16F10shPGC1α tumors normalized to B16F10shScrb1 tumors (t test, ns=not significant). FIG. 9H shows the relative PGC1α expression in neoplastic cells from resected tumors of patients with DCIS (n=5) and IDC categorized based on bone marrow metastases positivity (BM+: positive bone marrow metastases, n=12, BM−: no bone marrow metastases, n=13). Data is represented as mean+/−SEM.

### DETAILED DESCRIPTION

The present invention relates to methods, compositions, and diagnostic tests for treating and diagnosing a metastatic disease that results in increased mitochondrial respiration and/or biogenesis. In particular, the methods and compositions include treatment of metastatic diseases such as breast cancer using an antagonist of mitochondrial respiration such as a PGC1α antagonist.

Evaluating the metabolic requirement of migratory cancer cells in relation to proliferating cancer cells of primary tumors could be of infinite therapeutic value. In this regard, we show that the PGC1α-mediated bioenergetic switch to enhance mitochondrial respiration in cancer cells is functionally relevant for metastatic dissemination.

Invasive cancer cells from primary tumors and circulating tumor cells (CTC) revealed enhanced mitochondrial biogenesis and ATP production, a feature of non-dividing migratory cells. The enhanced mitochondrial respiration/oxidative phosphorylation did not impact glycolytic and anabolic rates in the CTC, and did not affect cancer cell proliferation or primary tumor growth kinetics. PGC1α suppression significantly impaired mitochondrial biogenesis and oxidative phosphorylation, and dissemination of cancer cells into the circulation and to secondary sites. These results suggest that while invasive and migratory properties of cancer cells are functionally dependent on mitochondrial respiration, their proliferative and anchorage-free survival can occur in an oxidative phosphorylation-independent fashion. Collectively, our studies favor the notion that glycolysis and anabolic pathways primarily regulate cancer cell proliferation, while mitochondrial respiration may facilitate cancer cell motility and invasion.

### Examples

### Experimental Methods

Animal studies: Orthotopic (breast pad for 4T1 and MDA-MB0231 and subcutaneous for B16F10) and intravenous (i.v.) injections of cancer cells were performed as previously described (Cooke et al., Cancer Cell 21:66-81, 2012; O'Connel et al., PNAS 108:16002-16007, 2011). MMTV-PyMT mice were previously described (Guy et al., Mol Cell Biol 12:954:961, 1992) and disease progression in these mice and experimental endpoint at which BCC, LCC and CTC was determined as previously described (Cooke et al., Cancer Cell 21:66-81, 2012). Metastatic surface area was computed as previously described (Cooke et al., Cancer Cell 21:66-81, 2012). Blood volume collection to harvest CTC was 200 μl. Blood was incubated with ACK lysis buffer (2 ml/200 μl blood for 15 minutes at 4° C.) before FACS purification based on GFP expression. For CTC colony formation, ACK lysis buffer treated 200 μl blood was plated in cm² dishes in DMEM tissue culture media supplemented with 10% FBS and penicillin/streptomycin.

Cell lines, stable transfection of shPGC1a and over-expression of PGC1a: 4T1 (mouse breast adenocarcinoma), B16F10 (mouse melanoma), and LLC (mouse Lewis Lung adenocarcinoma), MDA-MB-231 (human breast adenocarcinoma), SW480 (human colon adenocarcinoma) and A549 (human lung adenocarcinoma) cell lines were obtained from ATCC and cultured in recommended tissue culture media. Partial gene mutations reported for these lines are listed below (WT: wild-type: no mutation; * known mutations): 4T1 (P53*)¹², B16F10 (P53^{WT}/Kras^{WT}/cMyc^{WT})²⁷, LLC (P53*)²⁸, MDA-MB-231 (P53*/Kras*/cMyc*)²⁹, SW480 (P53*/Kras*/cMyc^{WT})²⁹ and A549 (P53^{WT}/Kras*/cMyc^{WT})²⁹. For stable transfection of PGC1α, pre-designed shRNAs from Origene were used and puromycin resistant clones subsequently propagated. For over-expression of PGC1α, recombinant adenovirus expressing PGC1α was kindly provided by Dr. Bruce Spiegelman, Dana-Farber Cancer Institute, Boston, Mass.). For proliferation rate, cells growing exponentially were counted twice at 12 hour intervals and doubling rate calculated. Measurements were repeated three times and data show the average of all experiments. Gene expression array and real-time PCR validation: Relative mtDNA content measurements:

Measurement of oxygen consumption rate: RNA was extracted from BCC, LCC and CTC using RNeasy Plus Mini Kit (Qiagen) and submitted to the Molecular Genetics Core Facility at Children's Hospital (Boston, Mass.). Microarray analysis was performed using Mouse Ref8 Gene Expression BeadChip (Illumina platform) and Metacore (GeneGo) and Knowledge Based Pathway (IPA) (rank invariant normalization with subtracted background, p<0.05). Gene expression validation by real-time PCR was performed as previously described (Cooke et al., Cancer Cell 21:66-81, 2012) using the primers listed in Table 1. The gene expression array data was deposited in Gene Expression Omnibus database (accession number GSE37344). Heat maps were drawn using R software.

TABLE 1

| Gene | Sequence |
|---|---|
| mo β-actin | F 5′-GGCTGTATTCCCCTCCATCG-3′ |
| | CCAGTTGGTAACAATGCCATGT-3′ |
| mo PGC1α | F 5′-AGCCGTGACCACTGACAACGAG-3′ |
| | R 5-GCTCATGGTTCTGAGTGCTAAG-3′ |
| mo NRF1 | F 5′-CTGCTGTCTCTTTCGGATAGATC-3′ |
| | R 5′-CGGAAACGGCCTCATCTCT-3′ |
| mo UCP1 | F 5′-GAGGTGTGGCAGTGTTCSTTG-3′ |
| | F-5′-GGCTGCATTGTGACCTTCA-3′ |
| mo ERRα | F 5′-AGGAAGCCCCGATGGA-3′ |
| | R 5′-GAGAGGCCTGGGATGCTCTT-3′ |
| Mo Elovl6 | F 5′-AAGCAGTTCAACGAGAACGAA-3′ |
| | R 5′-CGTACAGCGCAGAAAACAGG-3′ |
| mo Cox5b | F 5′-GGAAGACCCTAATCTAGTCCCG-3′ |
| | R 5′-CCACTATTCTCTTGTTGCTGAT-3′ |
| mo Cox4i | F 5′-ATTGGCAAGAGAGCCATTTCTAC-3′ |
| | R 5′-CAACACTCCCATGTGCTCGAA-3′ |
| mo ATPsynth | F 5′-GAGACTGGGCGTGTGTTAG-3′ |
| (ATP5a1) | R 5′-CTCGACGCAATACCATCACCA-3′ |
| ma CytC | F 5′-AAAGGGAGGCAAGCATAAGAC-3′ |
| | R 5′-GAACAGACCGTGGAGATTTGG-3′ |
| mo ACC | F 5′-ATGGGCGGAATGGTCTCTTTC-3′ |
| (ACC265) | R 5′-TGGGGACCTTGTCTTCATCAT-3′ |
| mo FASN | F 5′-AGGTGGTGATAGCCGGTATGT-3′ |
| | R 5′-TGGGTAATCCATAGAGCCCAG-3′ |
| mo CK8 | F 5′-TCCATCAGGGTGACTCAGAAA-3′ |
| | R 5′-CCAGCTTCAAGGGGCTCAA-3′ |
| mo Twist | F 5′-CGGGAGTCCGCAGTCTTA-3′ |
| | R 5′-TGAATCTTGCTCAGCTTGTC-3′ |
| mo Snail | F 5′-TCCAAACCCACTCGGATGTGAAGA-3′ |
| | R 5′-TTGGTGCTTGTGGAGCAAGGACAT-3′ |
| ma αSMA | F 5′-GGCACCACTGAACCCTAAGG-3′ |
| | R 5′-ACAATACCAGTTGTACGTCCAGA-3′ |
| mc Ecad | F 5′-GAGCCTGAGTCCTGCAGTCC-3′ |
| | R 5′-TGTATTGCTGCTTGGCCTCA-3′ |
| ma Slug | F 5′-CACATTCGAACCCACACATTGCCT-3 |
| | R 5′-TGTGCCCTCAGGTTTGATCTGTCT-3 |
| hu β-globin | F 5′-AGGAGAAGTCTGCCGTTACTG-3′ |
| | R 5′-CTTCATCCACGTTCACCTTGC-3′ |
| hu PGC1α | F 5′-GCTTTCTGGGTGGACTCAAC-3′ |
| | R 5′-CTGCTAGCAAGTTTGCCTCA-3′ |
| hu NRF1 | F 5′-AGGAACACGGAGTGACCCAA-3′ |
| | R 5′-TGCATGTGCTTCTATGGTAGC-3′ |
| hu Cox5b | F 5′-ATGGCTTCAAGGTTACTTCGC-3′ |
| | F 5′-CCCTTTGGGGCCAGTACATT-3′ |
| hu Cox4i | F 5′-ACTACCCCATGCCAGAAGAG-3′ |
| | R 5′-TCATTGGAGCGACGGTTCATC-3′ |
| hu ATPsynth | F 5′-TGCAAGGAACTTCCATGCCTC-3′ |
| | R 5′-CGCCCAGTTTCTTCAAGATCAA-3′ |
| hu CytC | F 5′-CTTTGGGCGGAAGACAGGTC-3′ |
| | R 5′-TTATTGGCGGCTGTGTAAGAG-3′ |
| hu ACC | F 5′-TGAGACTAGCCAAACAATCTCGT-3′ |
| | R 5′-AGAAAGTAGAAGCTCCGATCCT-3′ |
| hu FASN | F 5′-AAGGACCTGTCTAGGTTTGATGC-3′ |
| | R 5′-TGGCTTCATAGGTGACTTCCA-3′ |
| hu Elovl6 | F 5′-AGCAGTCAGTTTGTGACCAGG-3′ |
| | R 5′-ATCTCCTAGTTCGGGTGCTTT-3′ |
| hu Ecad | F 5′-CGAGAGCTACACGTTCACGG-3′ |
| | R 5′-GGGTGTCGAGGGAAAAATAGG-3′ |
| hu Twist | F 5′-CGGGAGTCCGCAGTCTTA-3′ |
| | R 5′-TGAATCTTGCTCAGCTTGTC-3′ |
| hu Snail | F 5′-GAGGCGGTGGCAGACTAG-3′ |
| | R 5′-GACACATCGGTCAGACCAG-3′ |
| hu αSMA | F 5′-GCTTTCAGCTTCCCTGAACA-3′ |
| | R 5′-GGAGCTGCTTCACAGGATTC-3′ |

ATP/ADP measurements: ATP/ADP measurements were obtained using the BioVision ApoSENSOR ADP/ATP Ratio Assay Kit according to the manufacturer's directions.

Targeted Mass Spectrometry Analysis: For cultured cells and FACS cells, 4 ml or 400 ml of 80% LC-MS grade methanol was added to each 10 cm² dish or FACS samples respectively and incubated at −80° C. for 15 minutes. Cells were scrapped and collected from plate to be centrifuged at full speed for 5 minutes in cold room to pellet cell debris and proteins. Supernatants were saved. Pellets were resuspended in 500 μl 80% methanol by vortexing and subsequently centrifuged like before. For cultured cells and FACS cells, 4 ml or 400 ml of 80% LC-MS grade methanol was added to each 10 cm² dish or FACS samples respectively and incubated at −80° C. for 15 minutes. Cells were scrapped and collected from plate to be centrifuged at full speed for 15 minutes at 4° C. to pellet cell debris and proteins. Supernatants were centrifuged one final time at 14,000 rpm for 10 minutes at 4° C. Metabolite extractions were dried to a pellet by SpeedVac with no heat. Samples were resuspended using 20 μL LC-MS grade water and 10 μL were injected and analyzed using a 5500 QTRAP hybrid triple quadrupole mass spectrometer (AB/Sciex) coupled to a Prominence UFLC HPLC system (Shimadzu) via selected reaction monitoring (SRM). 254 endogenous water soluble metabolites were targeted for steady-state analyses of samples. Some metabolites were targeted in both positive and negative ion mode via positive/negative polarity switching for a total of 289 SRM transitions. ESI voltage was +4900V in positive ion mode and 4500V in negative ion mode. The dwell time was 3 ms per SRM transition and the total cycle time was 1.56 seconds. Approximately 10-12 data points were acquired per detected metabolite. Samples were delivered to the MS via normal phase chromatography using a 4.6 mm i.d×10 cm Amide XBridge HILIC column (Waters) at 350 μL/min. Gradients were run starting from 85% buffer B (HPLC grade acetonitrile) to 35% B from 0-3.5 minutes; 35% B to 2% B from 3.5-11.5 minutes; 2% B was held from 11.5-16.5 minutes; 2% B to 85% B from 16.5-17.5 minutes; 85% B was held for 7 minutes to re-equilibrate the column. Buffer A was comprised of 20 mM ammonium hydroxide/20 mM ammonium acetate (pH 9.0) in 95:5 water:acetonitrile. Peak areas from the total ion current for each metabolite SRM transition were integrated using MultiQuant v2.0 software (AB/Sciex). Metabolomics data analysis was done in part using Metaboanalyst software (www.metaboanalsyst.ca<http://www.metaboanalsyst.ca>). For glucose isotopic tracer experiments, cells were placed in glucose-free media supplemented with 10% dialyzed serum and with uniformly labeled [U-¹³C₆] glucose (Cambridge Isotope Laboratories) for 12 hours before extraction for LCMS/MS analyses. A set of SRM transitions were used to target the heavy form of each metabolite.

Invasion and migration assays: For PGC1α gene expression analysis of collected cells directly following migration, uncoated polycarbonate membranes (8 μm pore) were used. The cells were seeded in the upper chamber and the migrated cells in the lower chamber were collected 12 hours following seeding. For invasion assays, the polycarbonate membranes were coated on both sides with Matrigel and cells on the basal side of the membrane (post migration) were fixed in 100% ethanol and stained with hematoxylin before microscopic evaluation. For hypoxia stimulation, the cells were stimulated for 4 hours prior to seeding into the Boyden chamber. For the scratch/migration assay, the cell free area was measured 24 hours after scratching the dish, and the experiment was done in triplicates.

Anoikis assay: 5·10⁶ cells are starved in 0.5% FBS for 24 hours. The cells are then counted and resuspended in 13 ml serum free DMEM in 15 ml Falcon tube and allowed to rock at 37° C. for 24 hours. The cells are then pelleted and counted using a hemocytometer. The two cell counts are used to determine the percent viability.

Type I collagen contractibility assay: 5·10⁴ cells/well of 24-well plates were seeded on 3 mg/ml type I collagen gel. Stressed matrix is allowed to contract for 48 hours and released. Collagen gel size change (average gel area) was measured with a ruler 24 hours following release of stressed matrix.

FACS: Tumors were resected, minced, and digested in 400 U/ml type II collagenase at 37° C. while shaking. Single cell suspension following filtering through 75 mm mesh were fixed in BD Cytofix/Cytoperm (BD Biosciences) and stained in 2% FBS containing PBS with DMEM with anti mouse αSMA antibody and TRITC conjugated secondary antibody. All FACS analyses were performed at the Joslin Diabetes Center Flow Cytometry Core, Boston, Mass. FACS purified cells were spun down at 5,000 rpm for 10 minutes at 25° C. and cell pellet processed for QPRC analysis using Cells-to-cDNA kit (Ambion) according to the manufacturer's direction.

Immunostaining: Thin frozen sections (5 μm) were immunolabeled and quantitation of immunolabeling was performed as previously described (Cooke et al., Cancer Cell 21:66-81, 2012).

Western blot analyses: Western blot analyses were performed as previously described (Cooke et al., Cancer Cell 21:66-81, 2012), using anti-PGC1α antibody (Calbiochem 4C1.3, 1 μg/ml) as recommended by the manufacturer.

Patient information and data collection: Patients were diagnosed with breast cancer and tumors were surgically resected at the Department of Gynecology, University Medical Center Hamburg-Eppendorf (Hamburg, Germany). Written informed consent was obtained and the study was approved by the University Medical Center Hamburg-Eppendorf institutional review board. Material collection and processing was previously described (Woelfle et al., Cancer Res 63:5679-5684, 2003) and RNA from patients diagnosed with ductal carcinoma in situ (DCIS), and invasive ductal carcinoma (IDC, all early stage estrogen receptor responsive primary tumors) with bone marrow aspirate positivity characterized. Detection of disseminated tumor cells in bone marrow was performed with anti-cytokeratin monoclonal antibody A45-B/B3 as previously described (Braun et al., N Engl Med 342:525-533, 2000) and according to international standards (Fehm et al., Cancer 107:885-892, 2006). De-identified RNA samples from microdissected neoplastic cells from resected primary tumors were analyzed for PGC1α expression normalized to expression levels detected in DCIS patients. Details are provided in Table 2.

TABLE 2

| | Primary tumors | | | | | |
|---|---|---|---|---|---|---|
| | BM positive | | BM negative | | DCIS | |
| | n | % | n | % | n | % |
| Histology | | | | | | |
| Ductal | 13 | 92.9 | 16 | 100.0 | | |
| Other | 1 | 7.1 | 0 | 0.0 | | |
| Tumor stage | | | | | | |
| pT1 | 5 | 35.7 | 7 | 43.6 | | |
| pT2 | 9 | 64.9 | 9 | 56.2 | | |
| pT3 + 4 | | | | | | |
| Lymph node status | | | | | | |
| PN0 | 12 | 85.7 | 18 | 100.0 | | |
| pN positive | 2 | 14.3 | 0 | 0.0 | | |
| Metastatic status | | | | | | |
| M0 | 14 | 100.0 | 18 | 100.0 | | |
| M1 | 0 | 0.0 | 0 | 0.0 | | |
| Grade | | | | | | |
| GI | 1 | 7.1 | 0 | 0.0 | | |
| GII | 0 | 64.3 | 9 | 56.3 | | |
| GIII | 4 | 26.6 | 7 | 43.7 | | |
| Age | | | | | | |
| <50 | 3 | 21.4 | 5 | 31.2 | 1 | 20.0 |
| >50 | 11 | 76.6 | 11 | 68.7 | 4 | 80.0 |
| Hormone receptor | | | | | | |
| negative | 0 | 0.0 | 1 | 6.2 | | |
| positive | 14 | 100.0 | 18 | 93.8 | | |
| HER2 in prim. tum. | | | | | | |
| negative | 13 | 92.0 | 11 | 68.8 | | |
| positive | 1 | 7.1 | 5 | 31.2 | | |
| Relapse | | | | | | |
| no | 12 | 85.7 | 18 | 100.0 | 5 | 100.0 |
| yes | 2 | 14.9 | 0 | 0.0 | 0 | 0.0 |

Statistical analysis: For comparison between two groups, we performed a two-tailed unequal variance t test, and p<0.05 was considered statistically significant. Analysis of microarray data was performed using Metacore (GeneGo) and Knowledge Based Pathway (IPA) (p<0.05).

Circulating Tumor Cells (CTC) Exhibit Enhanced Mitochondrial Function and Oxidative Phosphorylation Associated with Elevated PGC1α Expression and Increased Mitochondrial Biogenesis

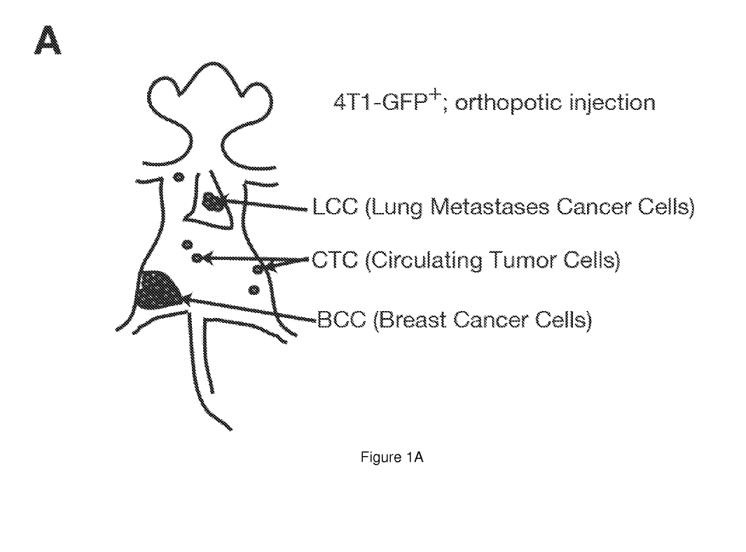

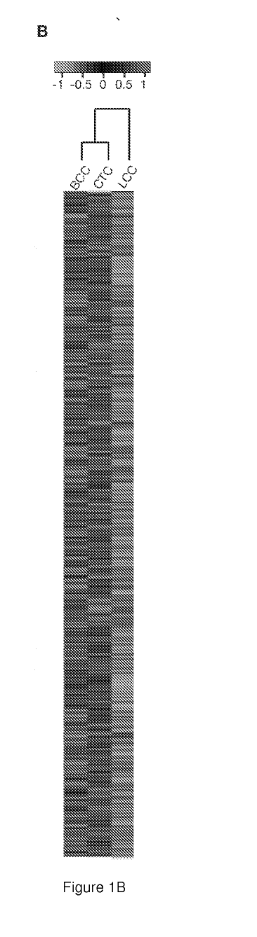

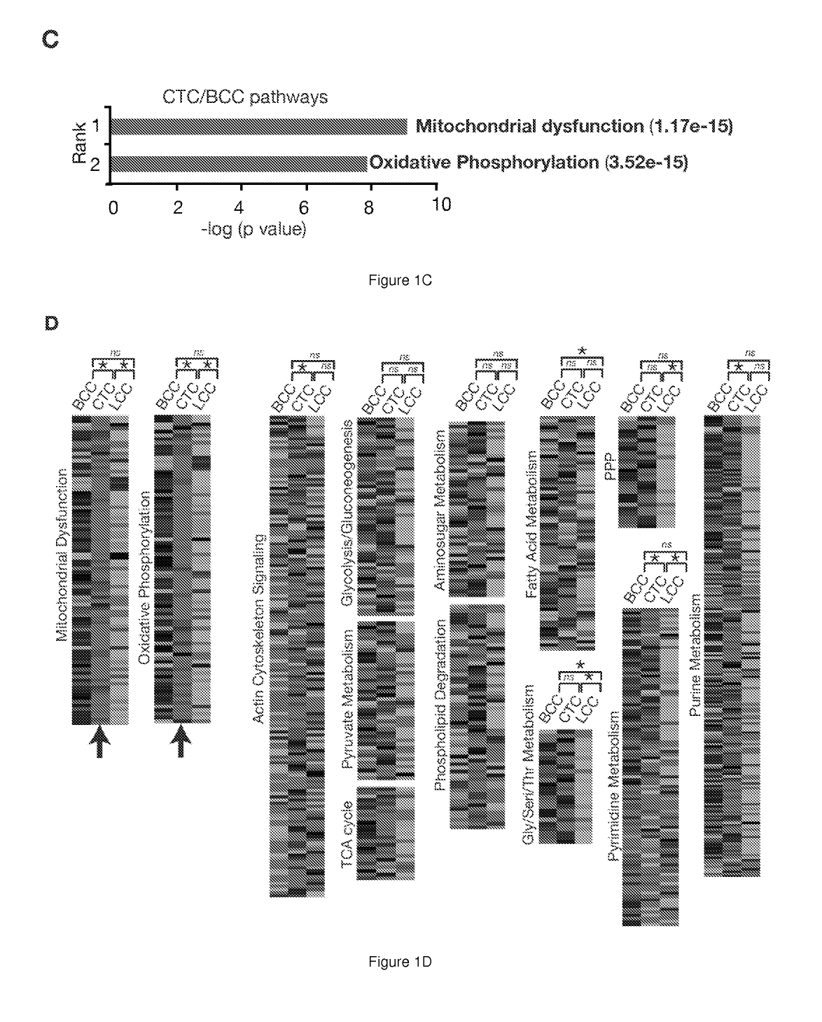

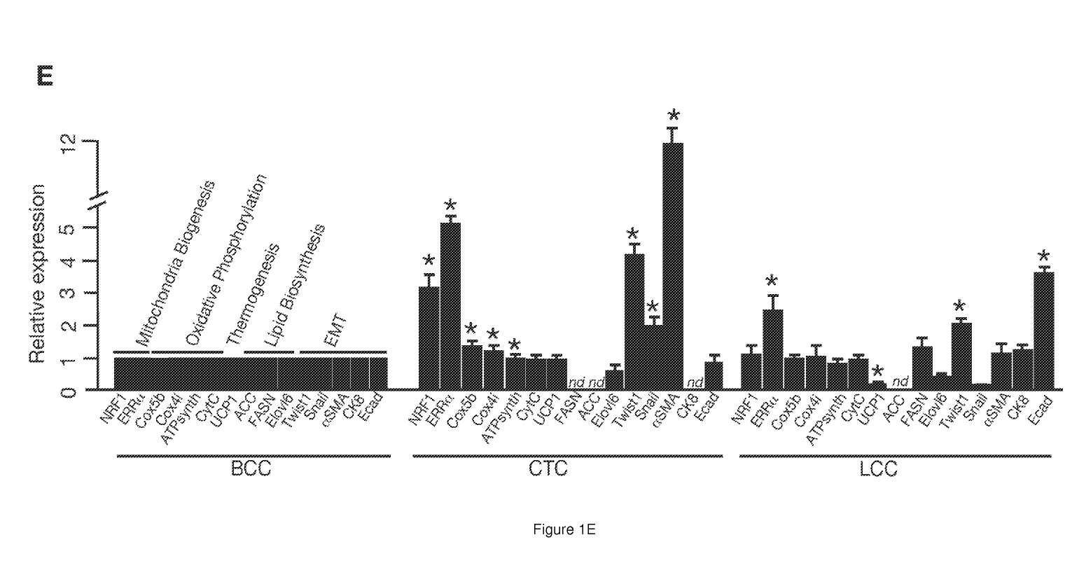

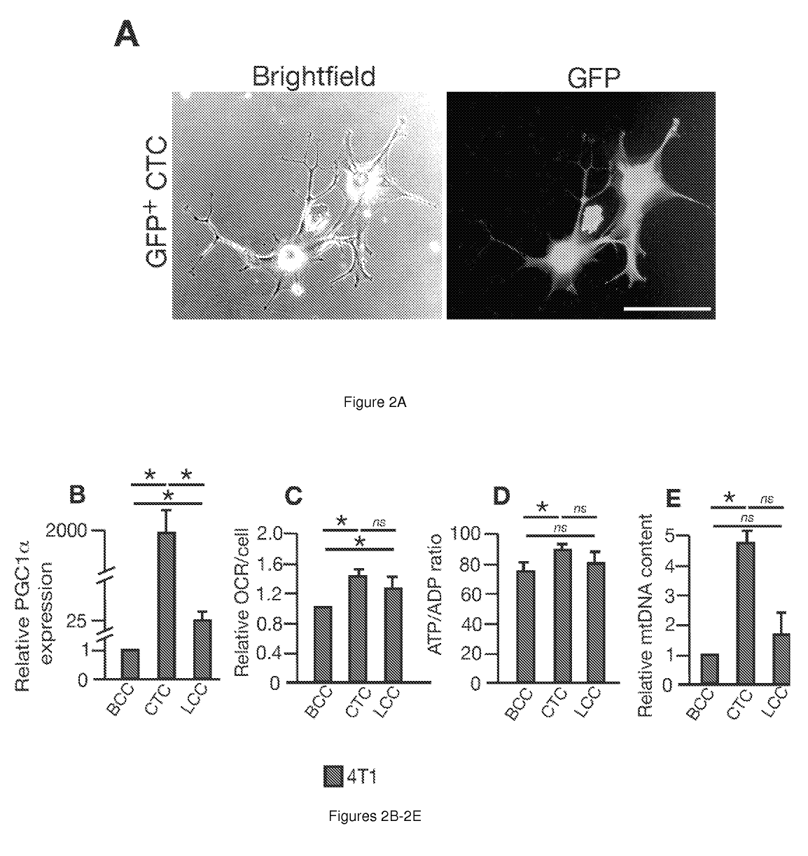

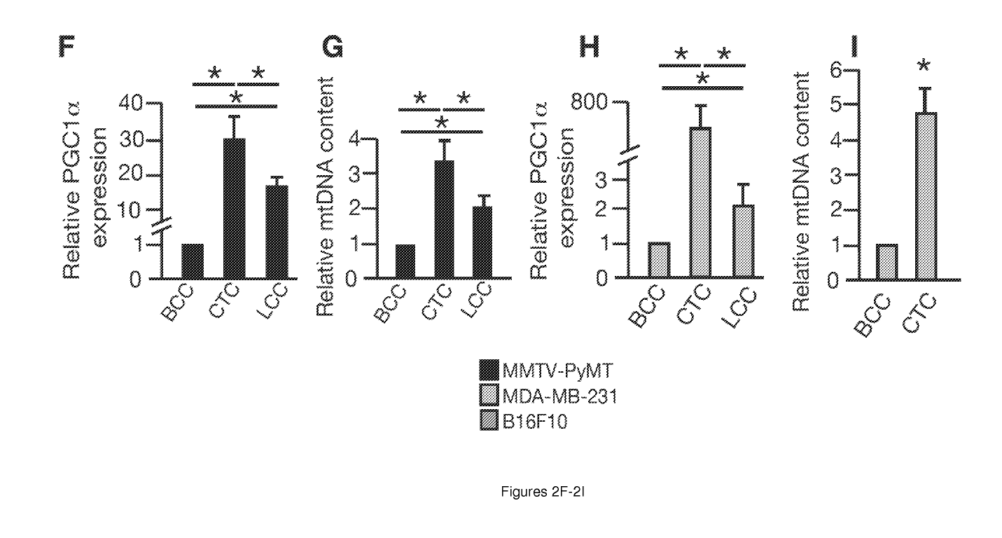

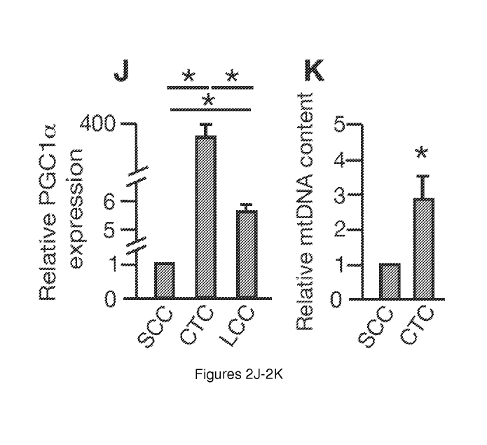

GFP-labeled 4T1 breast cancer cells were orthotopically implanted in the mammary pads of mice (FIG. 1A & FIG. 2A). In this mouse model for breast cancer, primary breast tumors emerge following injection of cancer cells in the breast pad of female mice and subsequently develop lung metastases with 100% penetrance. Circulating tumor cells (4T1-CTC) and cancer cells from the primary tumors (4T1 Breast Cancer Cells; 4T1-BCC) and metastatic lungs (4T1 Lung metastatic Cancer Cells; 4T1-LCC) were FACS purified and their transcriptome assayed by gene expression microarray (FIG. 1A). Heat map rendering of cluster analysis of their transcriptomes revealed that 4T1-CTC transcriptome differed from 4T1-BCC and 4T1-LCC transcriptome (FIG. 1B). Gene expression profiling coupled with bioinformatic analyses revealed that 4T1-CTC, when compared to 4T1-BCC, differentially express genes most significantly in the mitochondrial dysfunction and oxidative phosphorylation canonical pathways (the top two pathways) (FIG. 1C). Heat map rendering of the differentially expressed genes revealed a significant up-regulation of genes associated with mitochondrial function and oxidative phosphorylation in the 4T1-CTC (FIG. 1D). These genes were not differentially expressed in 4T1-BCC when compared to 4T1-LCC, suggesting a dynamic metabolic shift in 4T1-CTC that contrasts with both 4T1-BBC and 4T1-LCC. These findings prompted us to look at other metabolic pathways, including glycolysis/gluconeogenesis, pyruvate metabolism, TCA cycle, pentose phosphate pathway (PPP), amino-sugar metabolism, glycine/serine/threonine metabolism, fatty acid metabolism, and phospholipids degradation. These pathways were not differentially regulated in 4T1-CTC compared to 4T1-BCC (FIG. 1D). Genes associated with purine and pyrimidine metabolism were differentially expressed in 4T1-CTC compared to 4T1-BCC, as well as in 4T1-CTC compared to 4T1-LCC in the case of pyrimidine metabolism, perhaps reflecting feedback response resulting from altered oxidative phosphorylation in 4T1-CTC (FIG. 1D). Actin cytoskeleton signaling is upregulated in 4T1-CTC compared to 4T1-BCC and 4T1-LCC, further suggesting the unique and specific need for actin cytoskeletal rearrangement and derived signaling in migrating cancer cells (FIG. 1D). Q-PCR verified specific up-regulation of genes associated with mitochondrial biogenesis (NRF1, ERRc) and oxidative phosphorylation (Cox5b, Cox4i, ATPsynth, CytC) in 4T1-CTC compared to 4T1-BCC, while genes associated with thermogenesis or uncoupled respiration (UCP1) and lipid biosynthesis (ACC, Elov16, FASN) were unchanged (FIG. 1E). In contrast, 4T1-LCC showed a similar expression level of these genes when compared to 4T1-BCC (FIG. 1E), revealing a reversible expression profile of cancer cells that enter circulation to facilitate metastasis. This dynamic shift in metabolic gene expression pattern was also noticed with genes associated with an epithelial-to-mesenchymal (EMT) program. Mesenchymal genes (Twist, Snail, αSMA) were strongly upregulated in 4T1-CTC, while epithelial genes (CK8, Ecad) were downregulated in 4T1-CTC, compared to 4T1-BCC (FIG. 1E). The expression profile of these genes was similar in 4T1-LCC compared to 4T1-BCC (FIG. 1E). Together, these results indicate that 4T1-CTC present with enhanced mitochondrial function and oxidative phosphorylation in association with an EMT phenotype.

The acquisition of an enhanced mitochondrial oxidative phosphorylation in 4T1-CTC, when compared to 4T1-BCC and 4T1-LCC, was associated with a significant upregulation of PGC1α, an inducer of mitochondrial biogenesis, in 4T1-CTC and 4T1-LCC compared to 4T1-BCC, with very high level of expression detected specifically in the 4T1-CTC (FIG. 2A-B). PGC1α expression was not detected in the cells isolated from the blood of non-tumor bearing mice. Glycolysis/gluconeogenesis and lipid metabolism appeared unchanged in 4T1-CTC compared to 4T1-BCC and 4T1-LCC (FIG. 1D-E). 4T1-CTC however exhibited enhanced oxygen consumption rate (FIG. 2C), elevated ATP/ADP ratio (FIG. 2D), and increased mitochondrial DNA (FIG. 2E), indicative of mitochondrial biogenesis and respiration (FIG. 1C-D). These results suggested that the enhanced oxidative phosphorylation was associated with increased number of mitochondria per cell.

Elevated PGC1α expression and mitochondrial biogenesis was also observed in CTC from MMTV-PyMT transgenic mice, which spontaneously develop primary breast tumors that metastasize primarily to the lung (FIG. 2F-G), as well as in CTC from mice with MDA-MB-231 orthotopic metastatic breast tumors (FIGS. 2H-I), and CTC from mice with B16F10 metastatic melanoma tumors (FIG. 2J-K). These results suggested that the enhanced PGC1α expression and mitochondrial biogenesis was likely an important feature of CTC.

### PGC1α Expression Facilitates Mitochondrial Biogenesis and Invasion of Cancer Cells

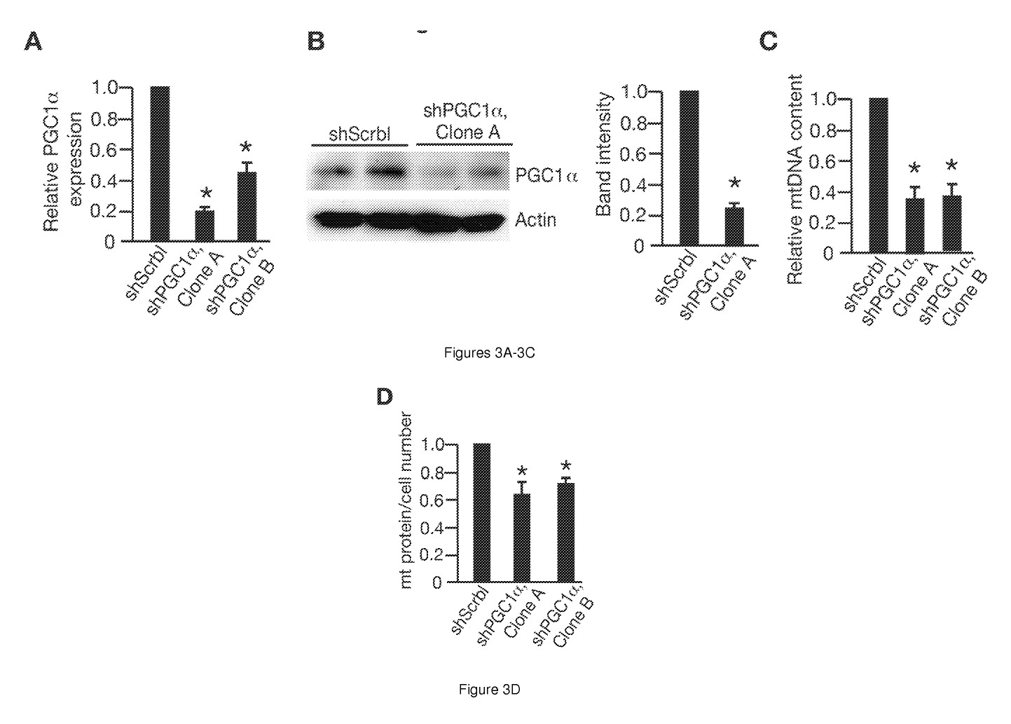

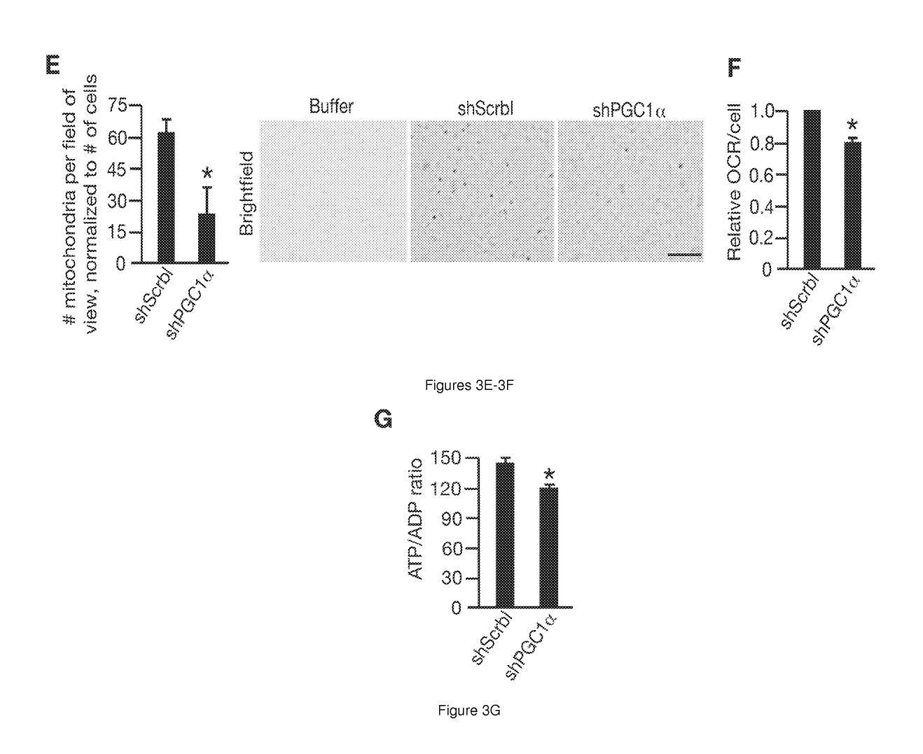

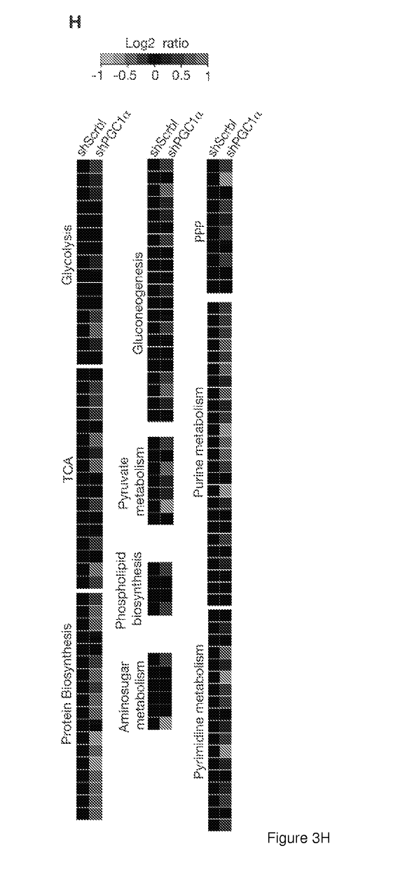

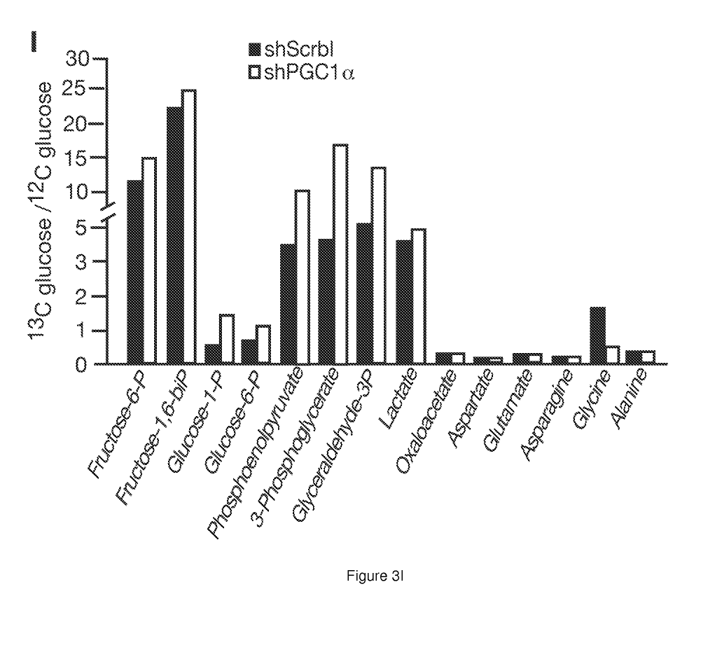

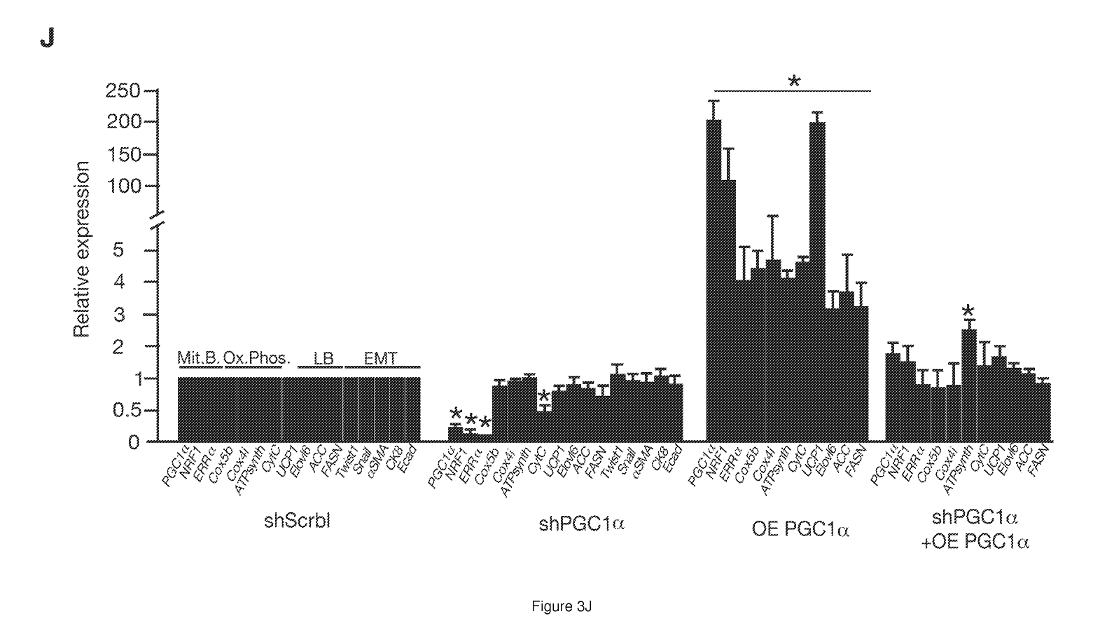

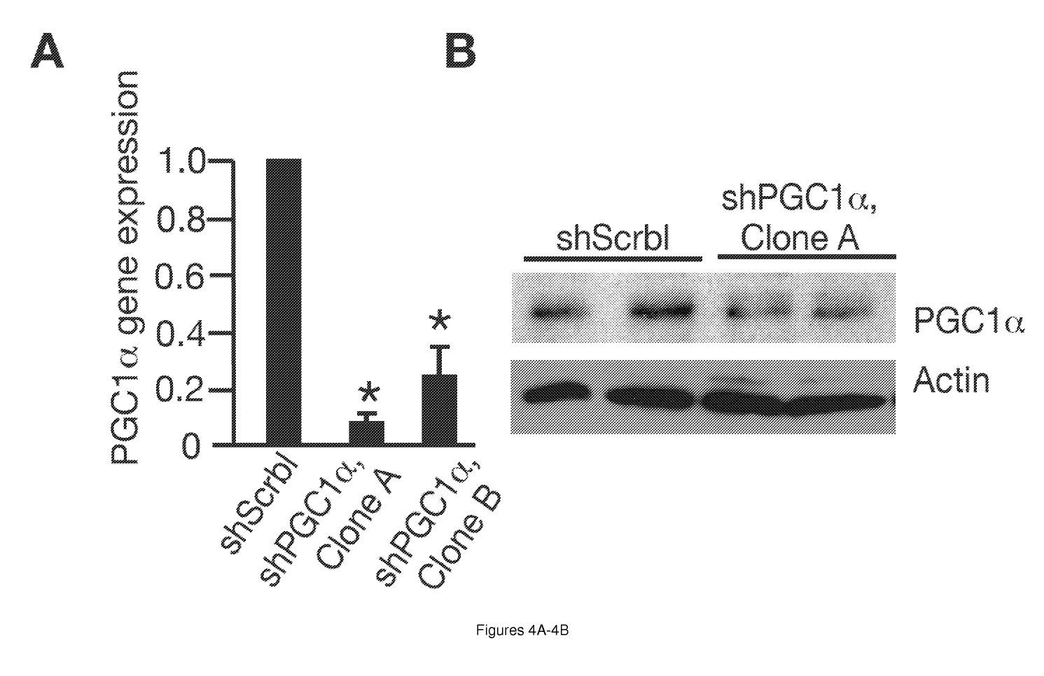

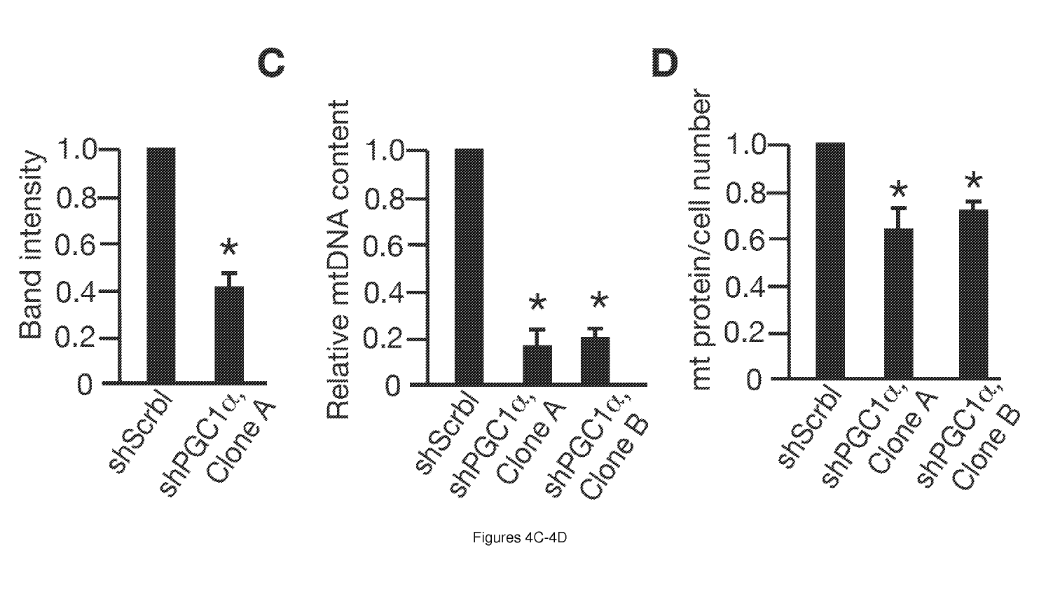

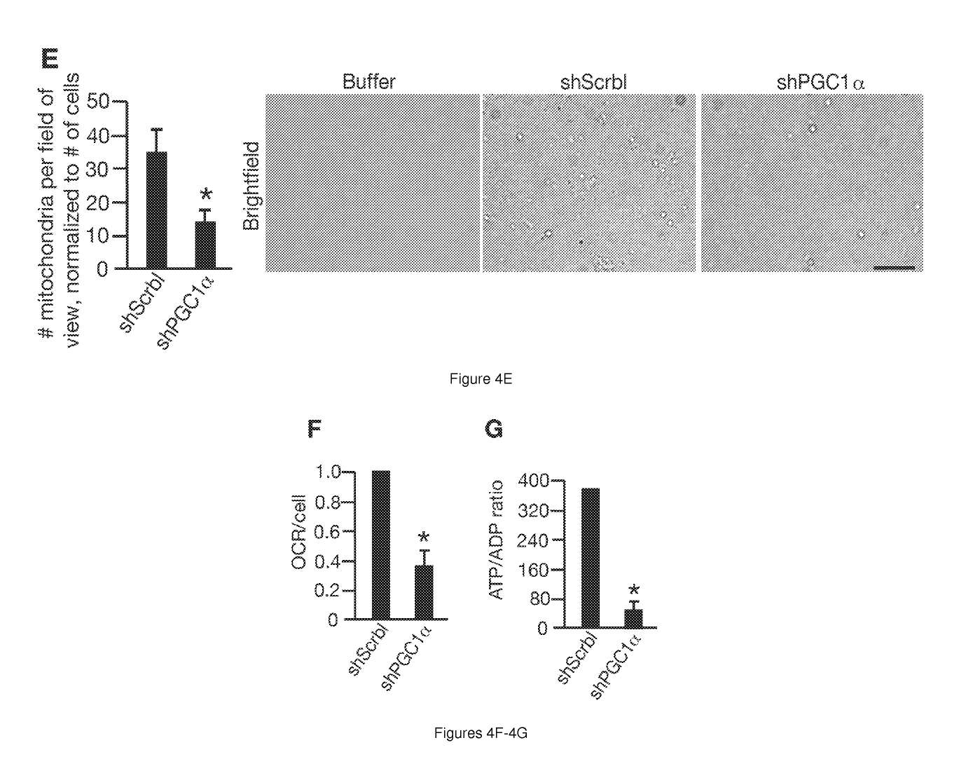

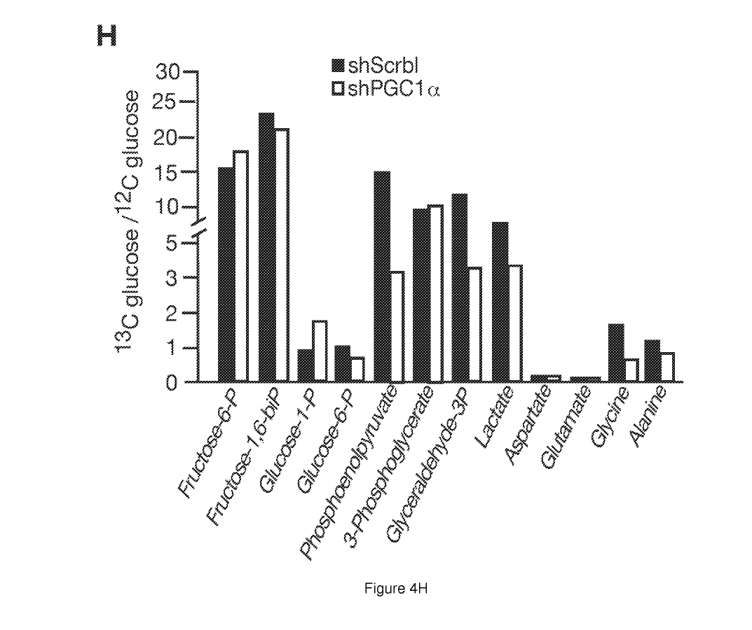

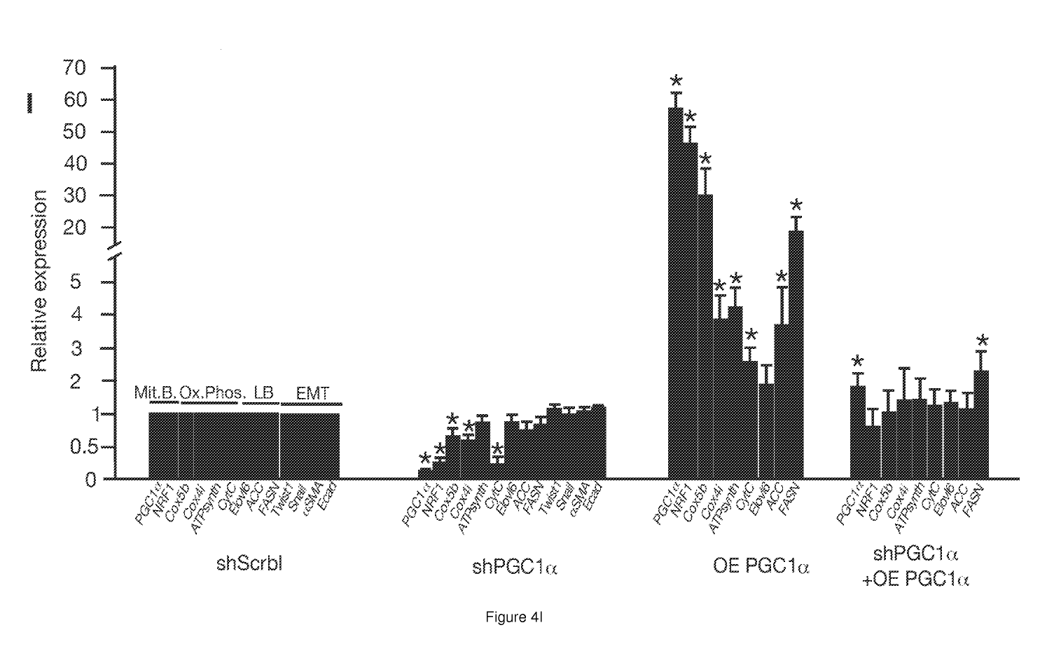

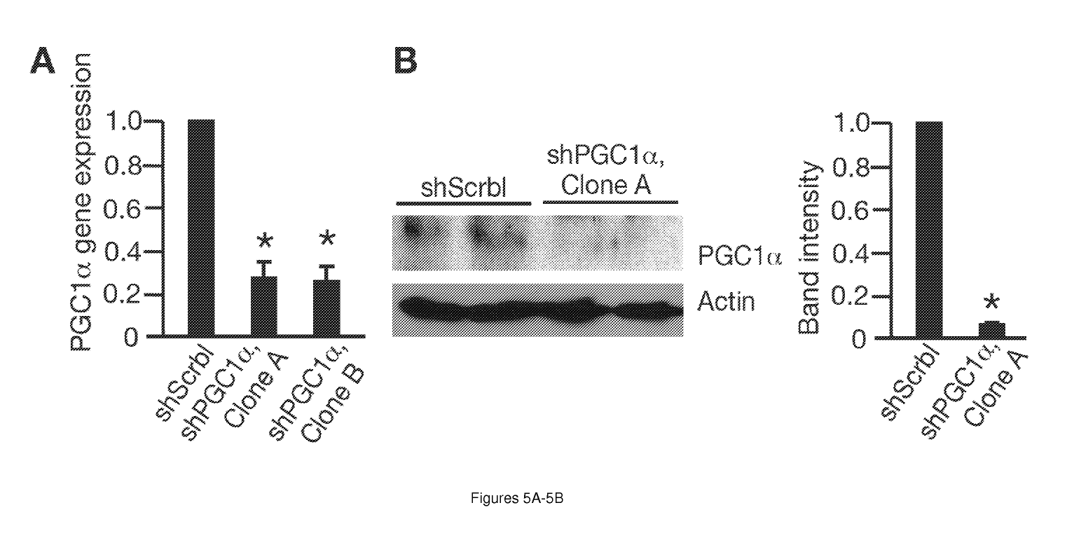

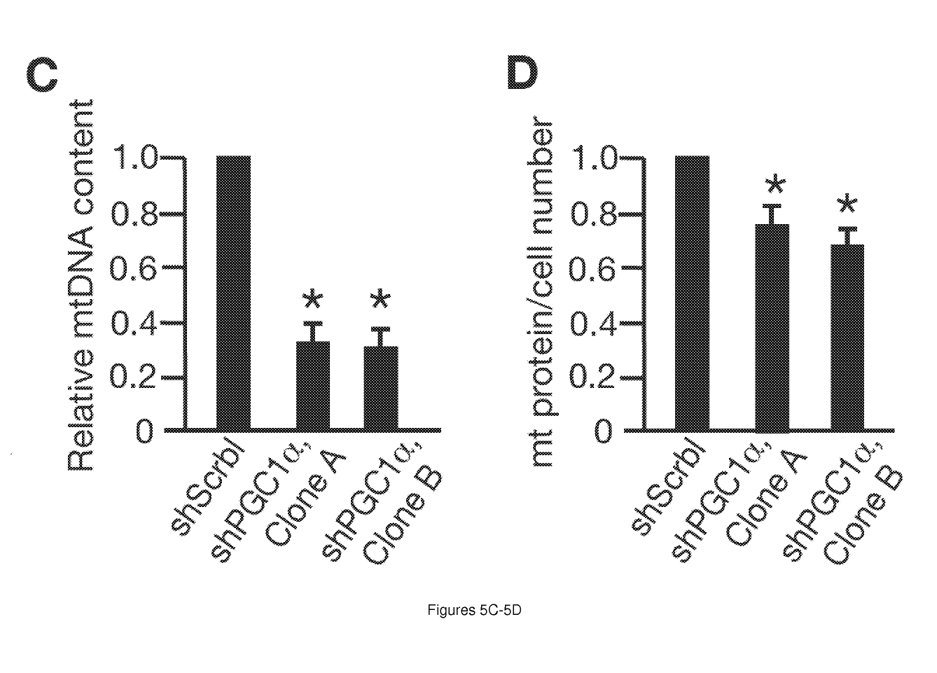

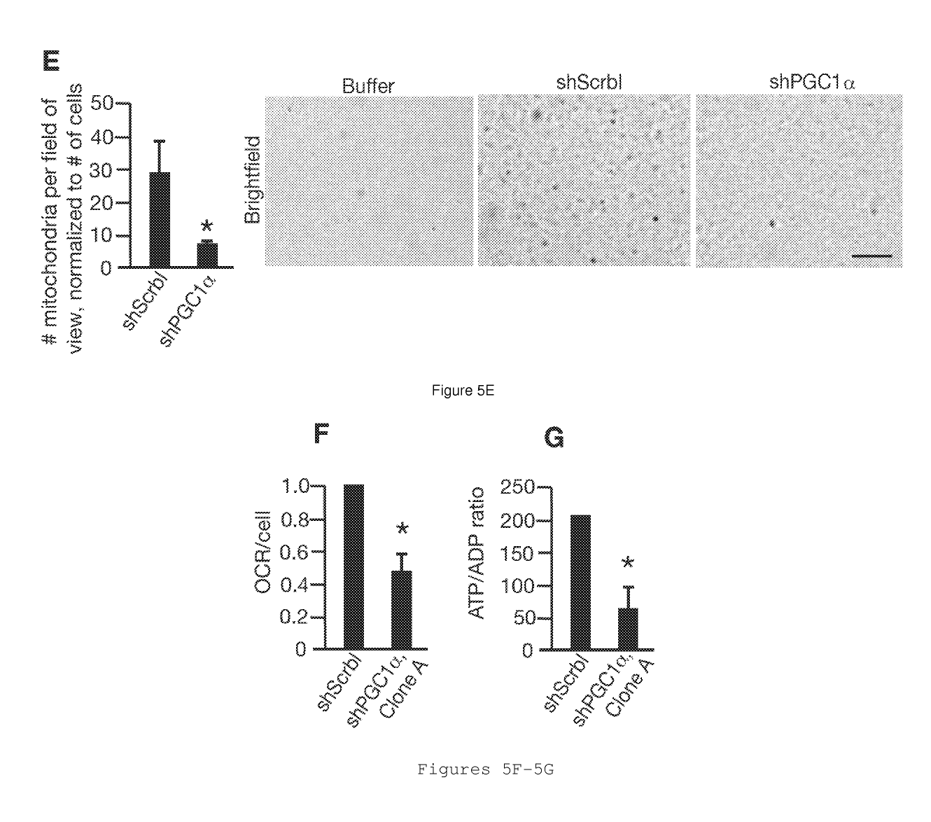

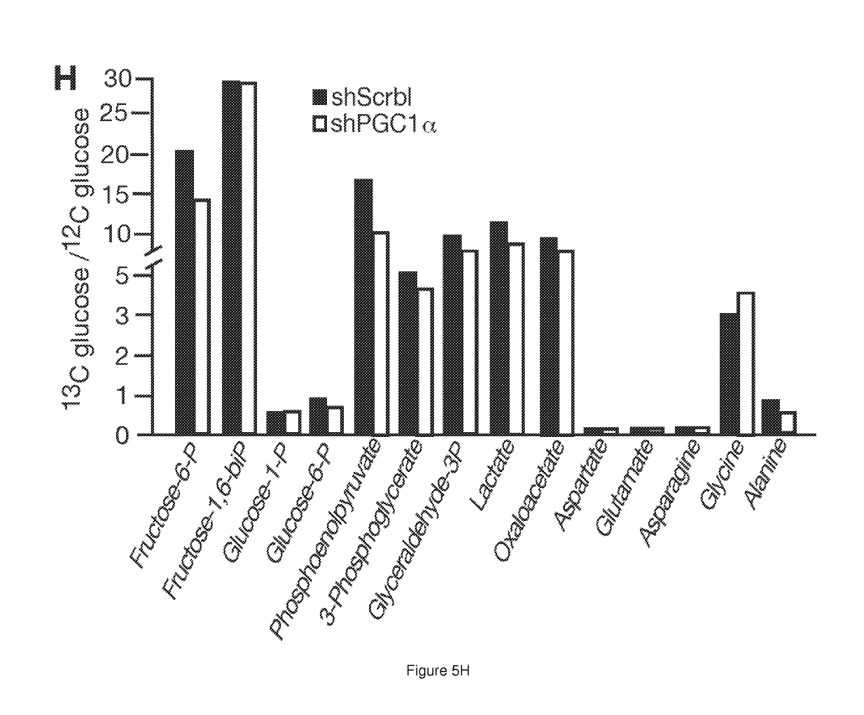

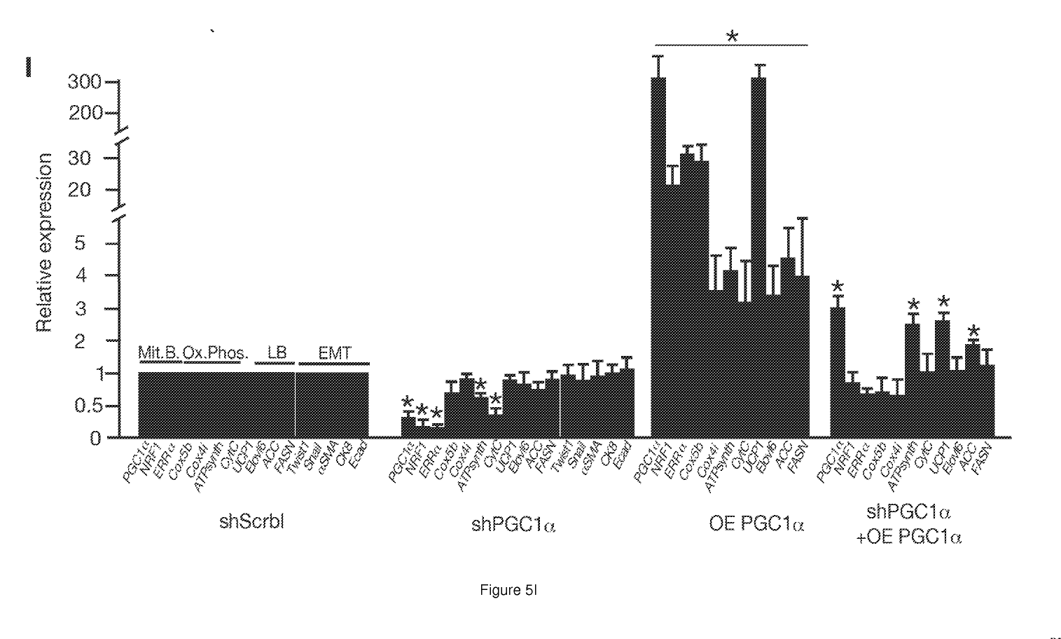

To determine the functional role of PGC1α in cancer cells, gene expression knockdown using shPGC1α and over-expression experiments were carried out to assess whether PGC1α and associated mitochondrial biogenesis/oxidative phosphorylation directly impact invasion and migration of cancer cells. First, PGC1α was silenced in 4T1 (metastatic mouse breast cancer), B16F10 (metastatic mouse melanoma) and MDA-MB-231 (metastatic human breast cancer) cells (FIG. 3-5). A significant reduction in PGC1α transcript and protein level in 4T1shPGC1α cells (FIG. 3A-B) resulted in suppressed mitochondrial biogenesis, as assessed by reduced mitochondrial DNA (FIG. 3C) and mitochondrial protein content per cell (FIG. 3D), when compared to control 4T1 cells (4T1shScrb1). Additionally, mitochondria number per cell was reduced in 4T1shPGC1α cells compared to 4T1Scrb1 cells (FIG. 3E), which together with reduced oxygen consumption rate and ATP production (FIG. 3F-G), indicated that suppression of PGC1α inhibited mitochondrial biogenesis and mitochondrial respiration in 4T1 cells. Similar findings were observed when PGC1α was suppressed in MDA-MB-231 and B16F10 cells (FIGS. 4A-G and 5A-G). Targeted mass spectrometry metabolomics analyses of 4T1shPGC1α compared to 4T1shScrb1 revealed insignificant impact on glycolysis, TCA cycle, gluconeogenesis, pyruvate metabolism, phospholipid biosynthesis, amino-sugar biosynthesis, pentose phosphate pathway (PPP), and purine/pyrimidine metabolism (FIG. 3H). These results suggested that suppression of PGC1α specifically reduced oxidative phosphorylation while pyruvate metabolism and TCA cycle were unaltered in cancer cells. Protein biosynthesis appeared downregulated in cells with suppression of PGC1α expression (FIG. 3H). Additionally, metabolomics analyses of 4T1shPGC1α cultured using labeled ¹³C-labeled glucose also showed only minor differences in accumulation of glycolytic/gluconeogenesis metabolites, lactate, oxaloacetate, and metabolites associated with protein and nucleotide biosynthesis when compared to 4T1shScrb1 cells (FIG. 3I). Q-PCR analyses supported reduced mitochondrial biogenesis/oxidative phosphorylation due to PGC1α gene suppression, with the significant down-regulation of genes associated with mitochondrial biogenesis (PGC1α, NRF1, ERRα), and oxidative phosphorylation (Cox5b, Cox4i, ATPsynth, CytC) in 4T1shPGC1α compared with 4T1shScrb1, while genes associated with lipid biosynthesis (ACC, Elov16, FASN) and EMT program (CK8, Ecad, Twist, Snail and αSMA) were unchanged (FIG. 3J). Adenoviral induction of PGC1α expression (over expression of PGC1α/OE PGC1α) reversed the suppression of genes associated with mitochondrial biogenesis and oxidative phosphorylation in 4T1shPGC1α cells while genes associated with lipid biosynthesis remained unchanged (FIG. 3J). Similar results attesting of shPGC1α-mediated suppression of mitochondrial respiration were also observed when B16F10 and MDA-MB-231 cells were used instead of 4T1 cells (FIGS. 4H-I and 5H-I). These results collectively suggest that PGC1α in this setting functions by modulating mitochondrial biogenesis while glucose metabolism pathways remain unaffected.

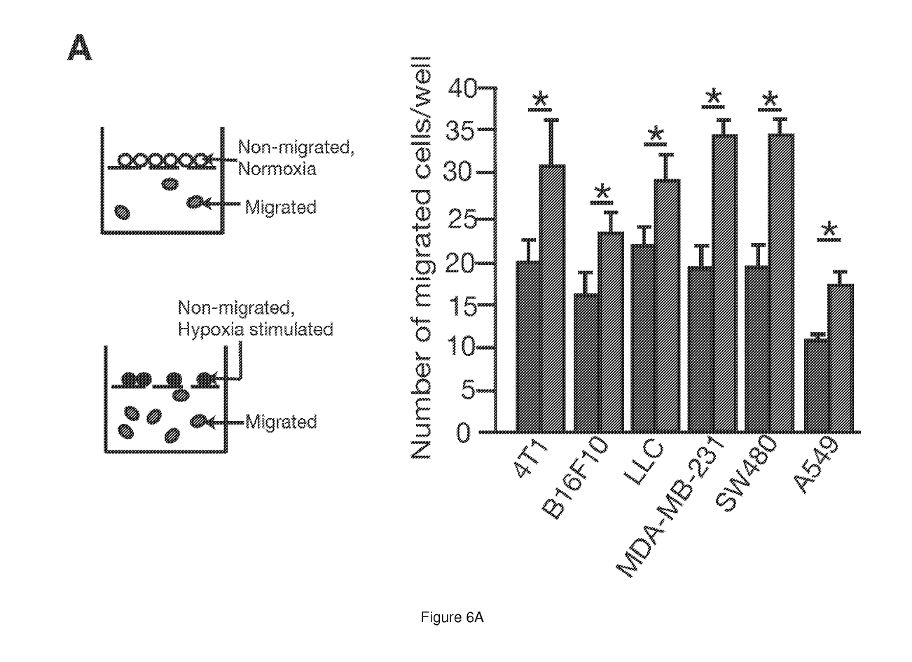

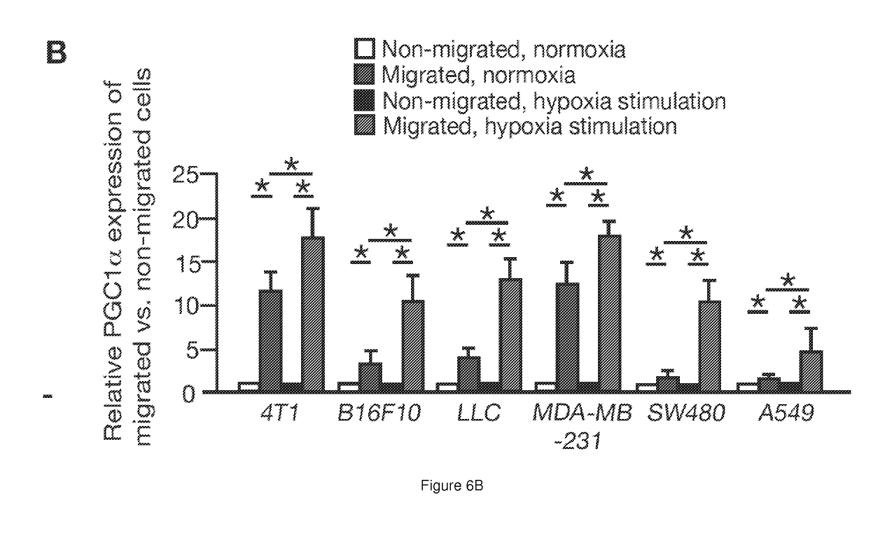

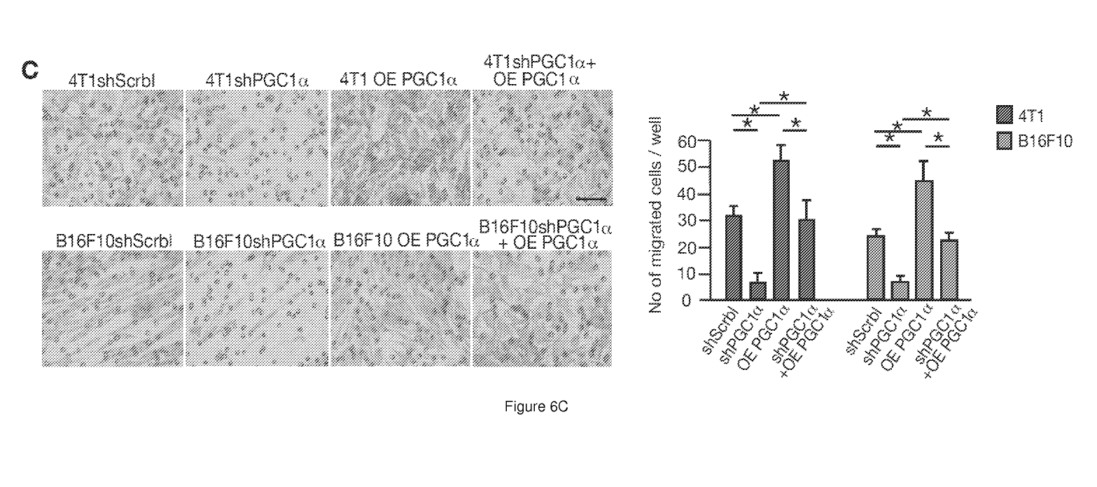

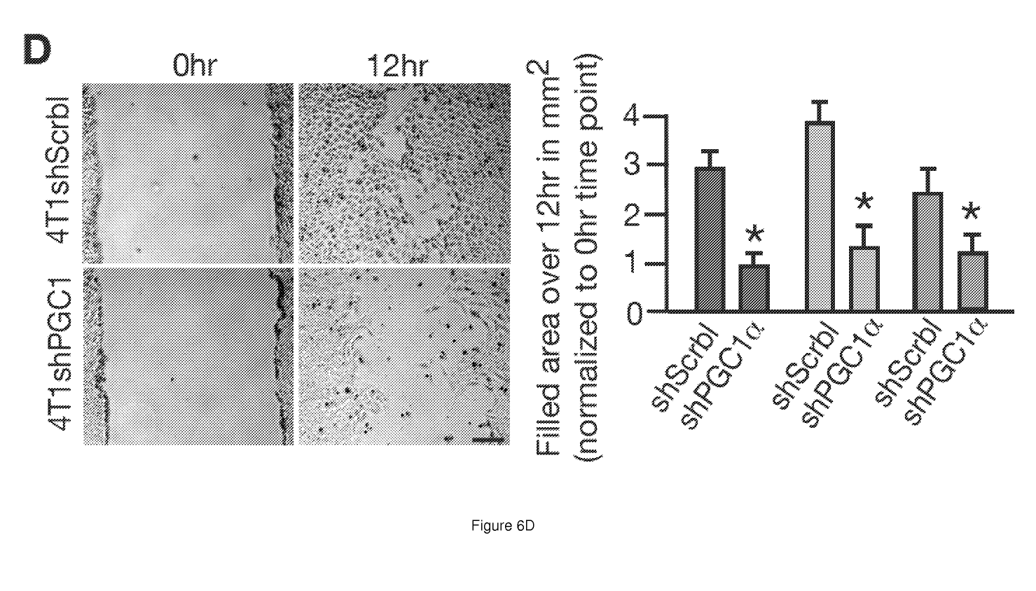

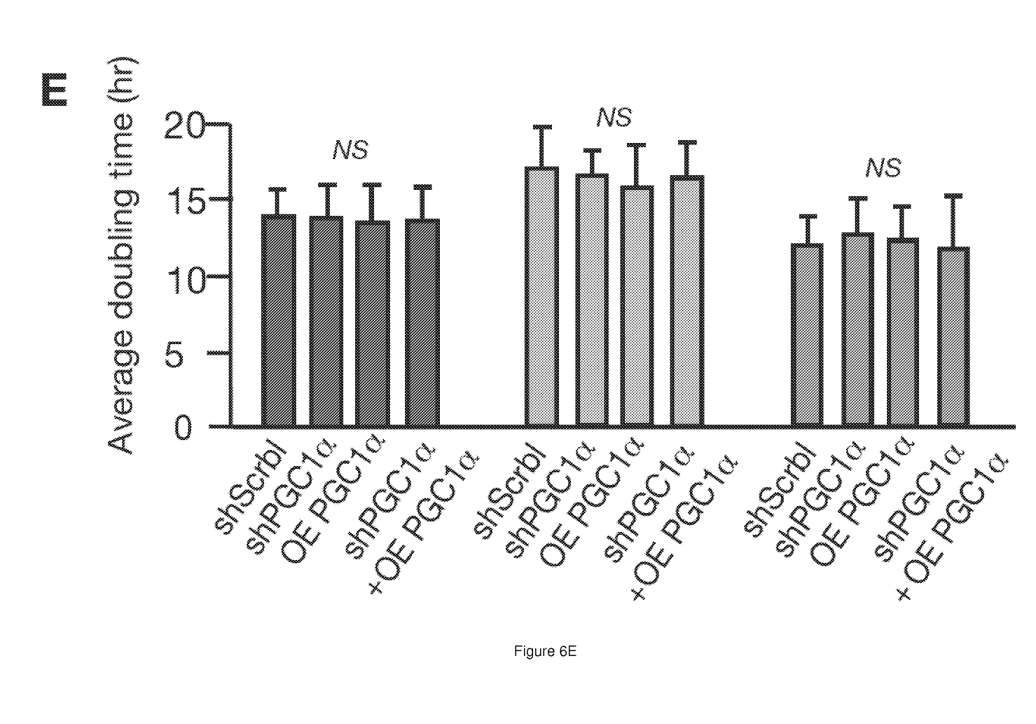

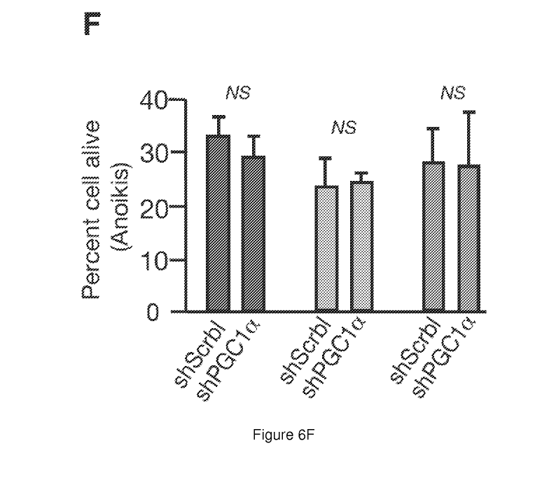

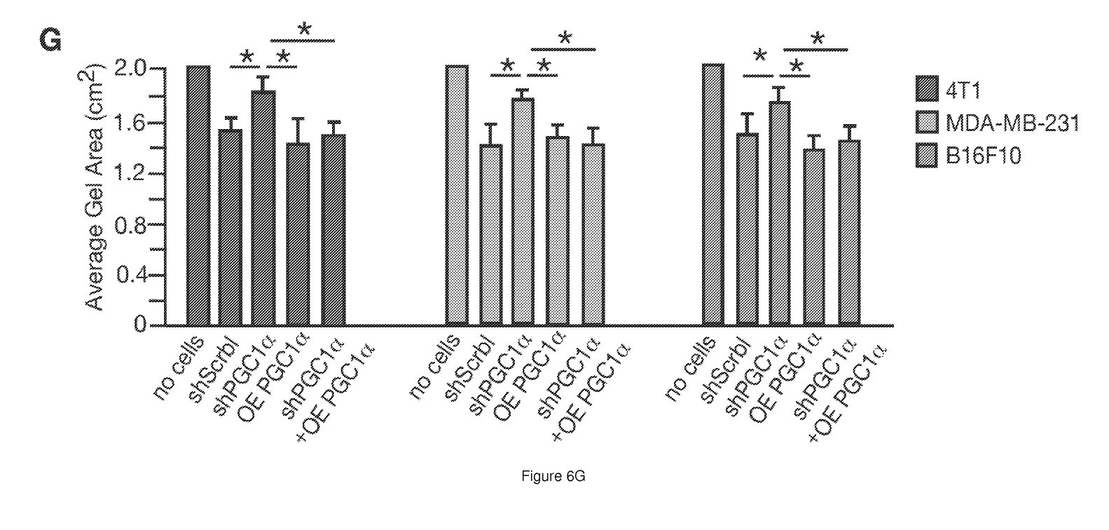

Since CTC are cells that have migrated away from the primary tumor and revealed increased expression of genes reflective of actin cytoskeleton signaling (FIG. 1D), we evaluated PGC1α expression in cancer cells following their migration in a Boyden chamber system with and without hypoxia. Hypoxia enhanced the migration of all six cell lines tested, mouse 4T1 (breast adenocarcinoma), B16F10 (melanoma), and LLC (Lewis lung adenocarcinoma), and human MDA-MB-231 (breast adenocarcinoma), SW480 (colon adenocarcinoma) and A549 (lung adenocarcinoma) (FIG. 6A). We evaluated migrated cancer cells versus non-migrated cancer cells that remained on the luminal side of the chamber. The migrated cancer cells showed enhanced PGC1α expression when compared to the majority of the adhered cells that did not migrate (FIG. 6B, green). All cell lines showed increased PGC1α expression associated with a migratory phenotype. Brief hypoxia stimulation of all cell lines enhanced their migration in association with a significant increase in PGC1α expression (FIG. 6B). We measured invasion, migration, proliferation and anchorage-independent survival of 4T1, B16F10 and MDA-MB-231 cells with stable down-regulation of PGC1α shPGC1α), over-expression of PGC1α or shPGC1α cancer cells with rescued PGC1α expression. While suppression of PGC1α revealed specific down-regulation of mitochondrial respiration, over expression of PGC1α in cancer cells resulted in mixed metabolic response, likely resulting from hyper induction of many metabolic processes due to high levels of PGC1α (FIGS. 3I-J, 4J and 5J). Nevertheless, over expression of PGC1α in shPGC1α cancer cells reversed mitochondrial biogenesis and respiration suppression (FIGS. 3I-J, 4J and 5J). While PGC1α knockdown significantly reduced invasion of cancer cells, over-expression of PGC1α enhanced invasion and rescued the reduced invasion observed in shPGC1α cells (FIG. 6C). Migration was similarly reduced in cancer cells with decreased PGC1α expression (FIG. 6D), while the rate of proliferation and anchorage-independent survival (anoikis) was unaffected by induced alterations in PGC1α expression (FIG. 6E-F). Loss of PGC1α expression reduced cancer cells' ability to tighten type I collagen in contraction assays, suggestive of compromised actin cytoskeleton structure (FIG. 6G). Over expression of PGC1α alone did not enhance collagen I contraction but the rescued shPGC1α cells restored type I collagen contractility (FIG. 6G). Taken together, our results indicate that loss of PGC1α expression diminishes invasive and migratory properties of cancer cells and such properties are restored with the rescue of PGC1α gene expression in the shPGC1α cells.

### PGC1α Facilitates Cancer Cell Dissemination and Metastasis

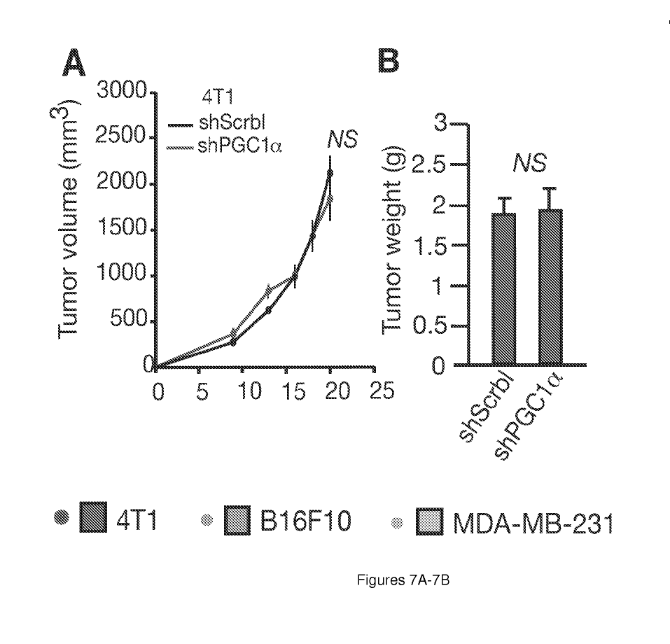

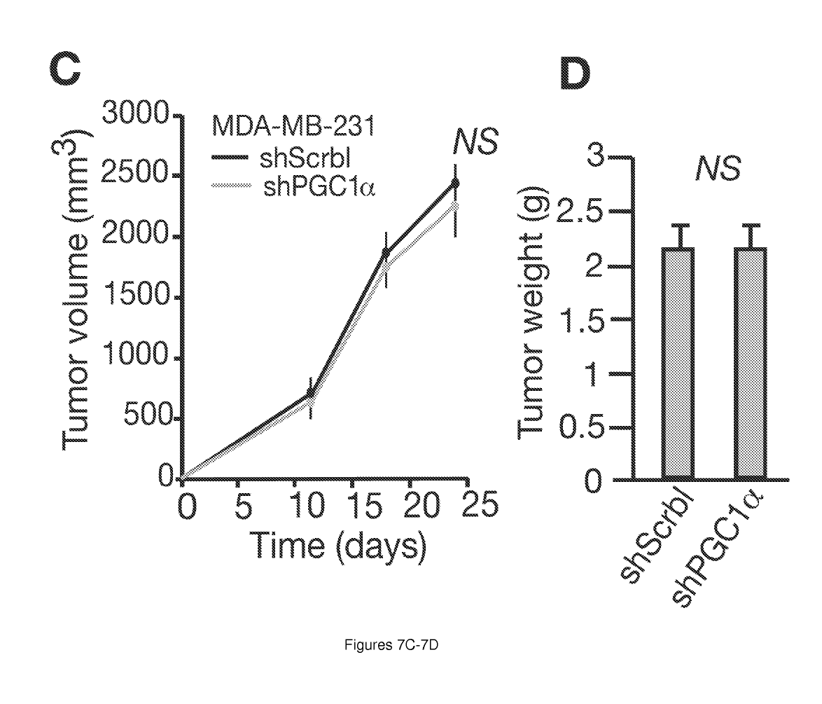

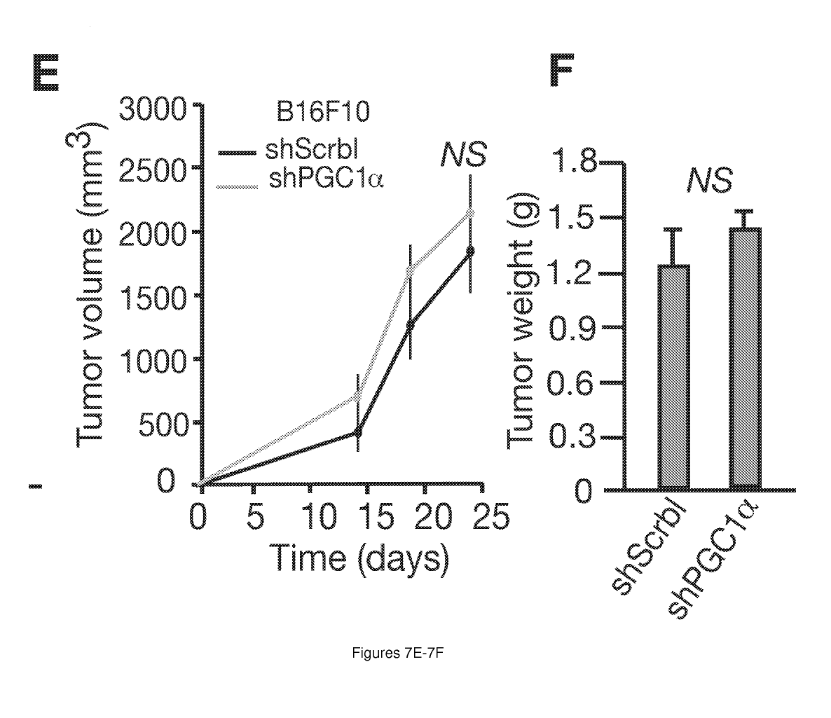

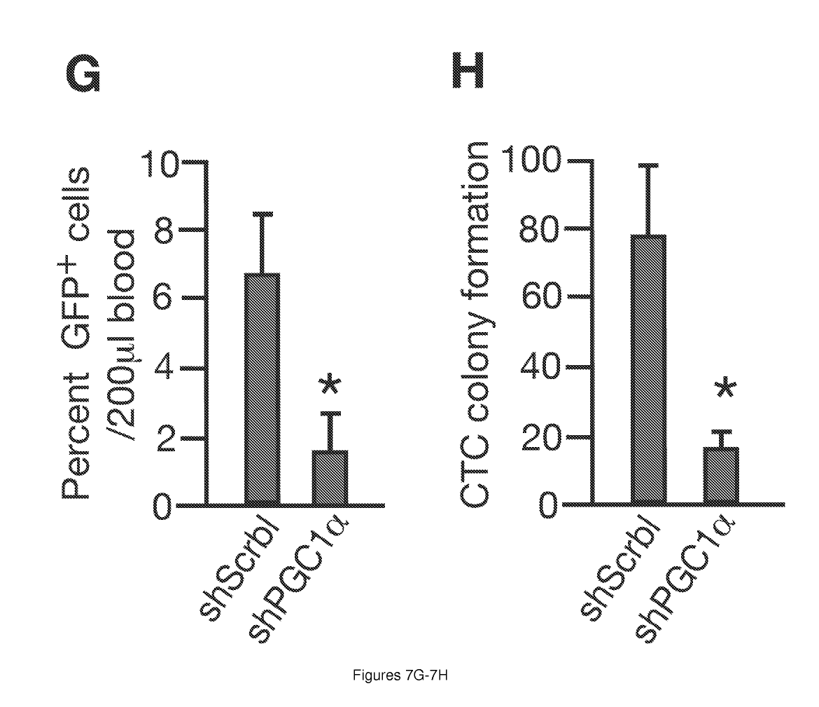

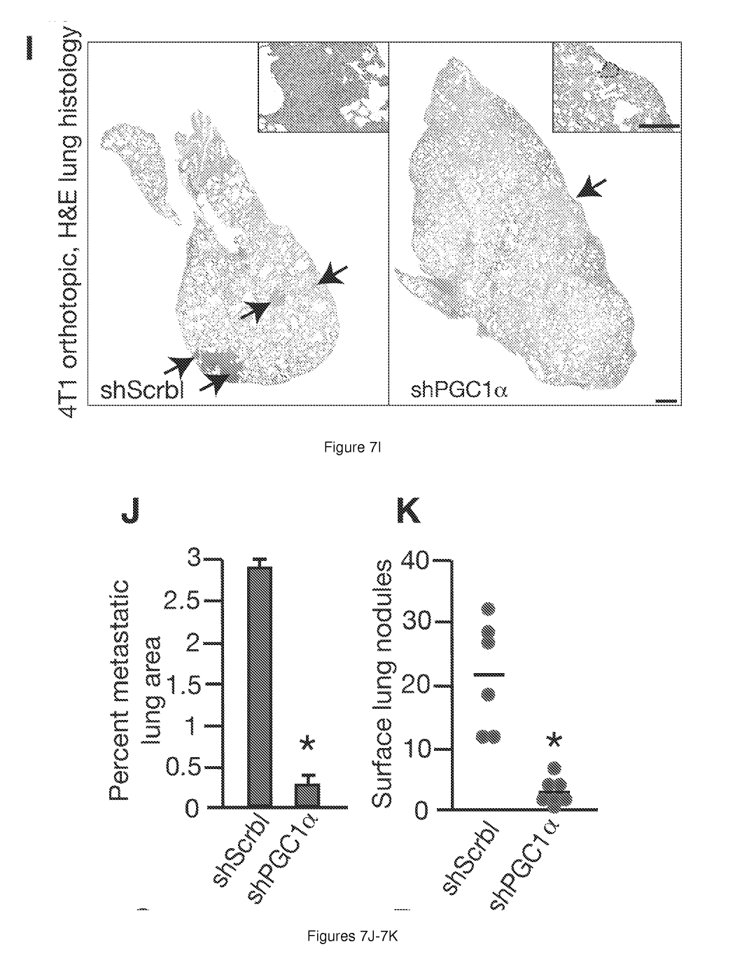

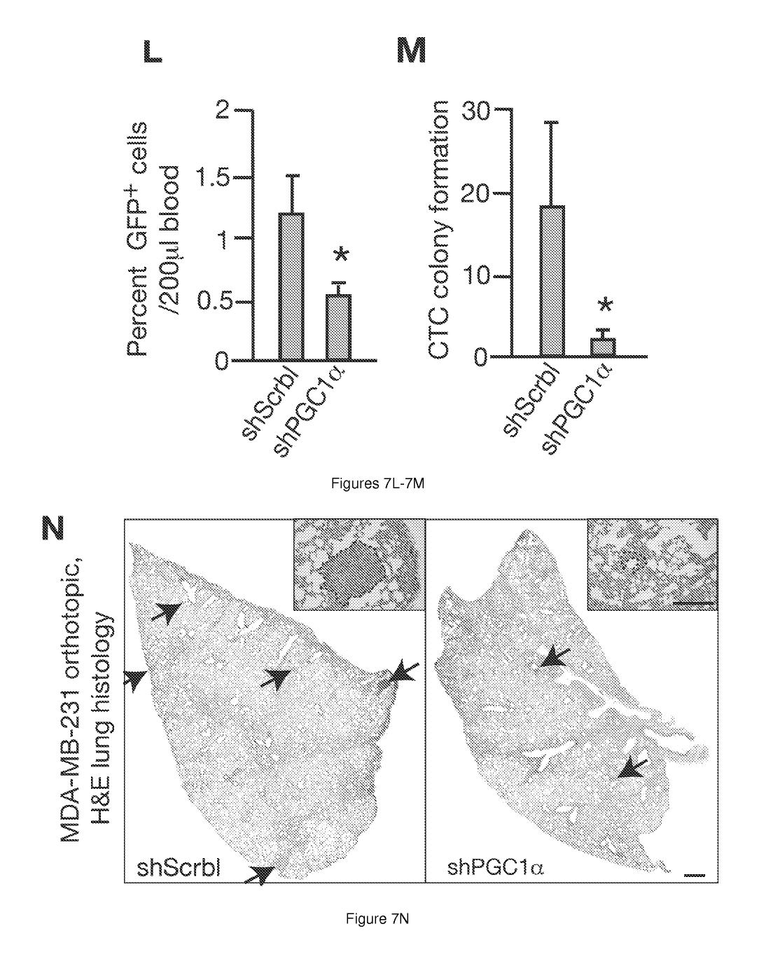

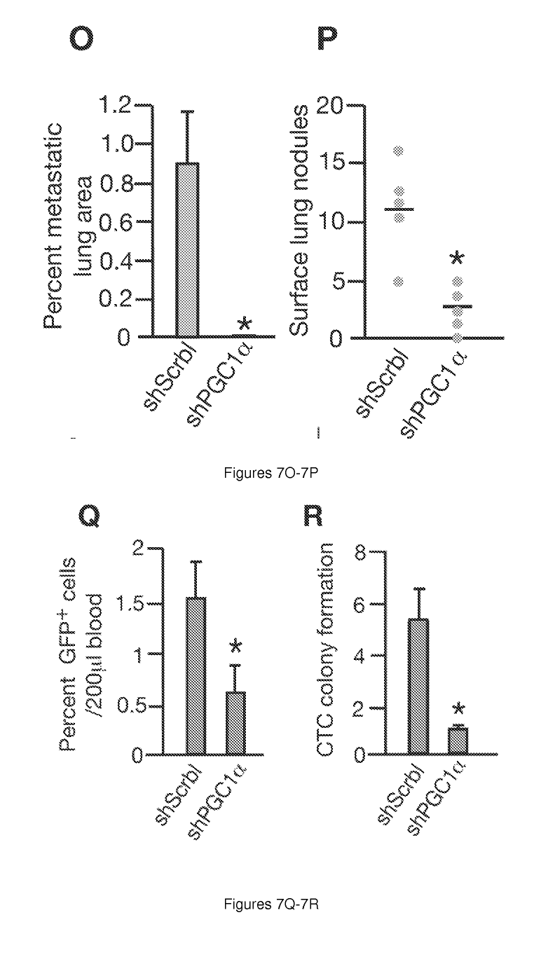

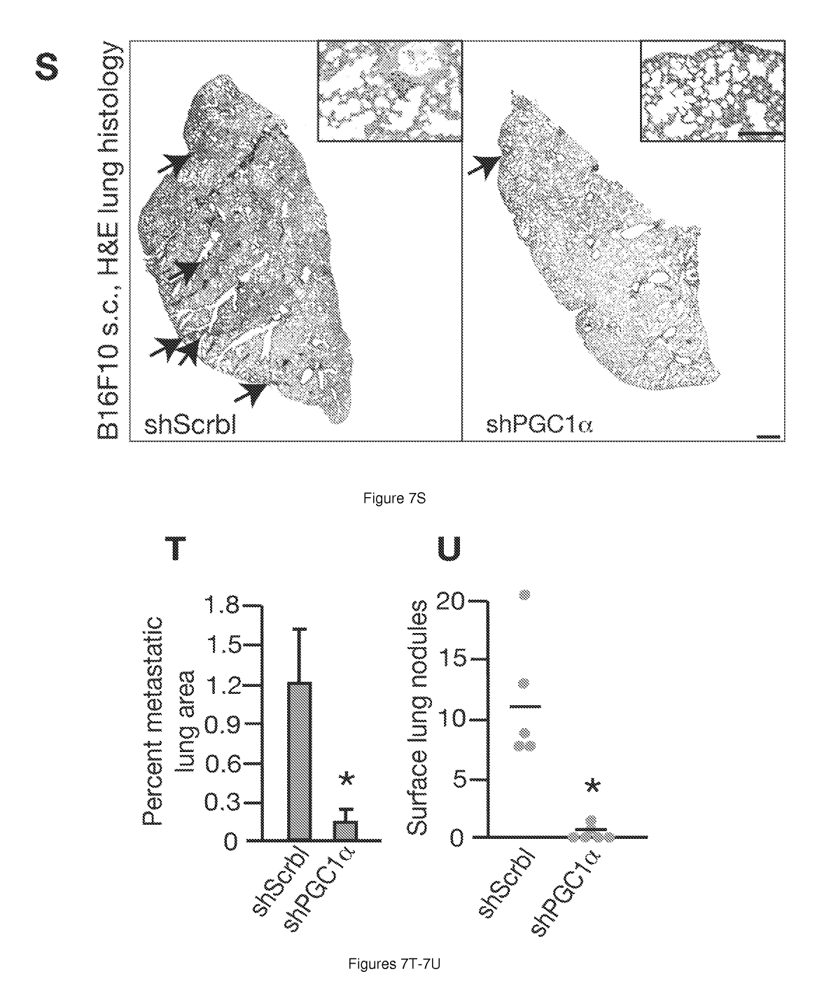

When implanted orthotopically, primary tumor growth kinetics of 4T1 cells with PGC1α gene expression knockdown (4T1shPGC1α) were similar to control 4T1 cells (4T1shScrb1) (FIGS. 7A and 7B). Proliferative index as measured by BrdU incorporation was unchanged, supporting the results obtained in vitro using the 4T1shPGC1α and 4T1shScrb1 cells (FIG. 6E). MDA-MB-231shPGC1α tumors showed similar tumor growth kinetics and weights compared to control MDA-MB-231shScrb1 tumors (FIGS. 7C and 7D). Similarly, PGC1α gene expression knockdown did not impact B16F10 primary tumor growth (FIG. 7E-F). The number of CTC was significantly reduced in mice with 4T1shPGC1α tumors compared to mice with control 4T1shScrb1, as assessed by the reduced percent GFP+ cancer cells in the blood by FACS analysis (FIG. 6G) and also by the decreased number of blood-derived cancer cell colonies (colony formation assay) (FIG. 7H). The decreased dissemination of cancer cells was associated with a significant reduction in the computed percent metastatic lung area and number of surface lung nodules of mice with 4T1shPGC1α tumors compared to mice with control 4T1shScrb1 tumors (FIG. 7I-K). All the above findings were reproduced using a second clone for the knockdown of PGC1α in 4T1 cells (FIG. 3A-D). CTC numbers (FIG. 7L-M) and metastasis (FIG. 7N-P) were also significantly reduced in mice bearing MDA-MB-231shPGC1α tumors in contrast with mice bearing MD-MB-231shScrb1 tumors. Decreased cancer cell dissemination and reduced metastatic disease were observed when PGC1α expression was suppressed in B16F10 melanoma cells (FIG. 7Q-U). These results suggest that in all three tumor models, suppression of PGC1α in cancer cells resulted in reduced dissemination of cancer cells and metastasis.

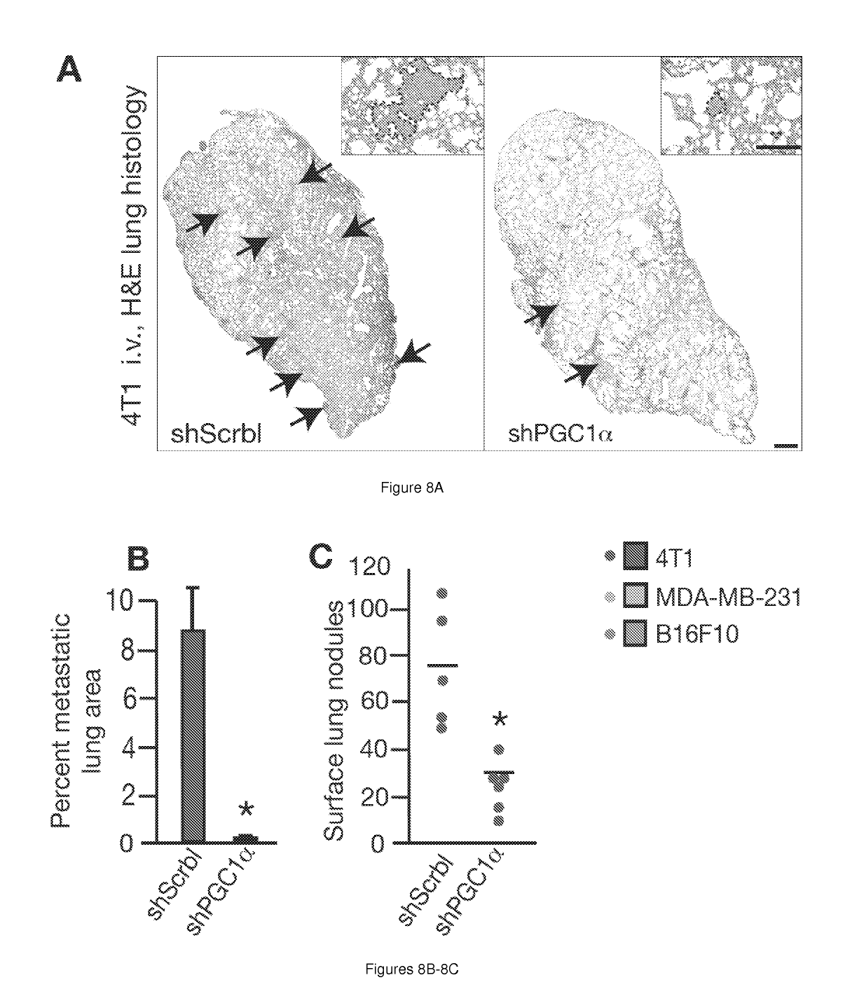

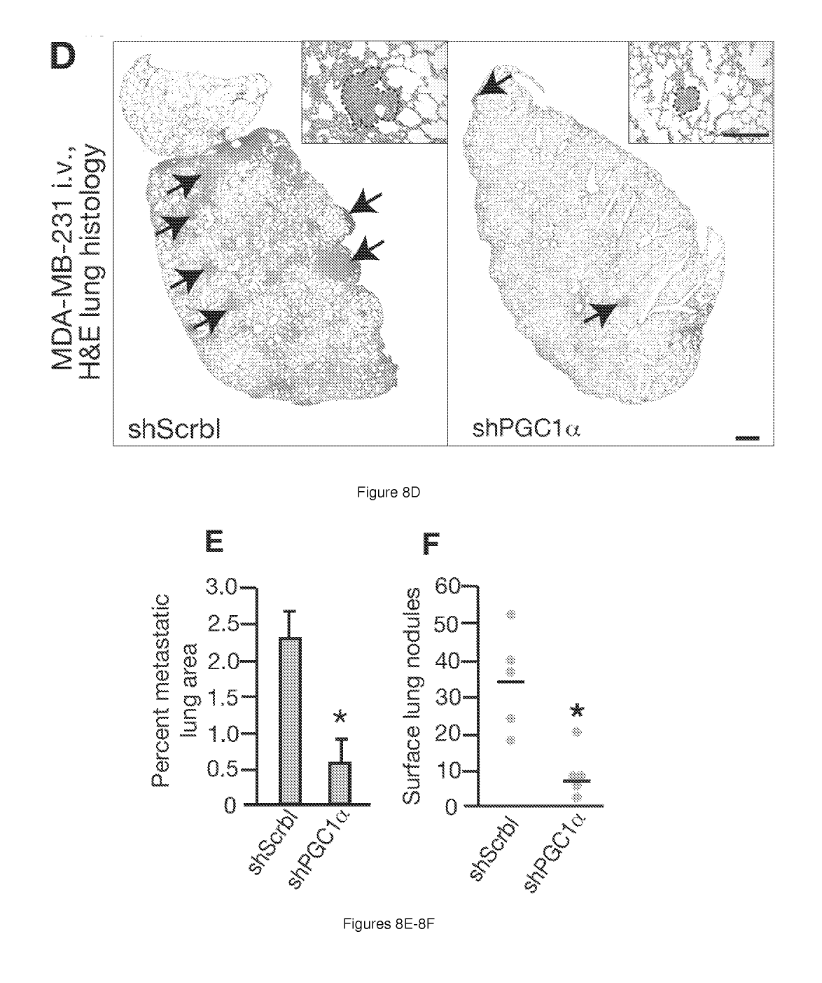

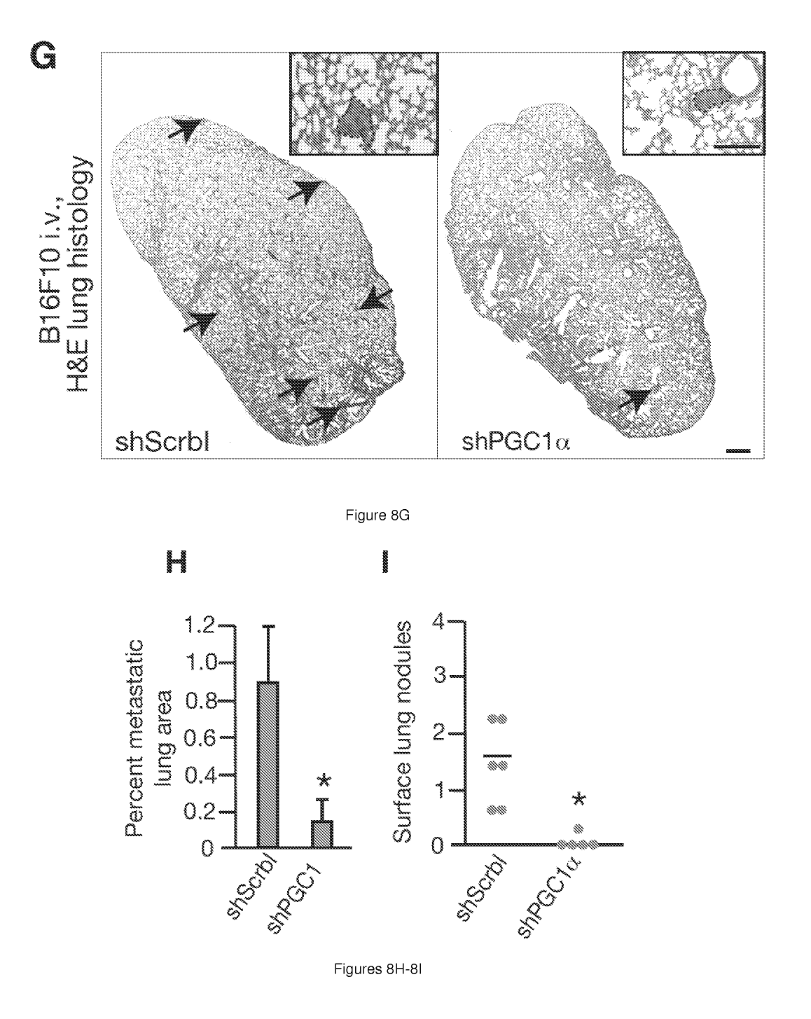

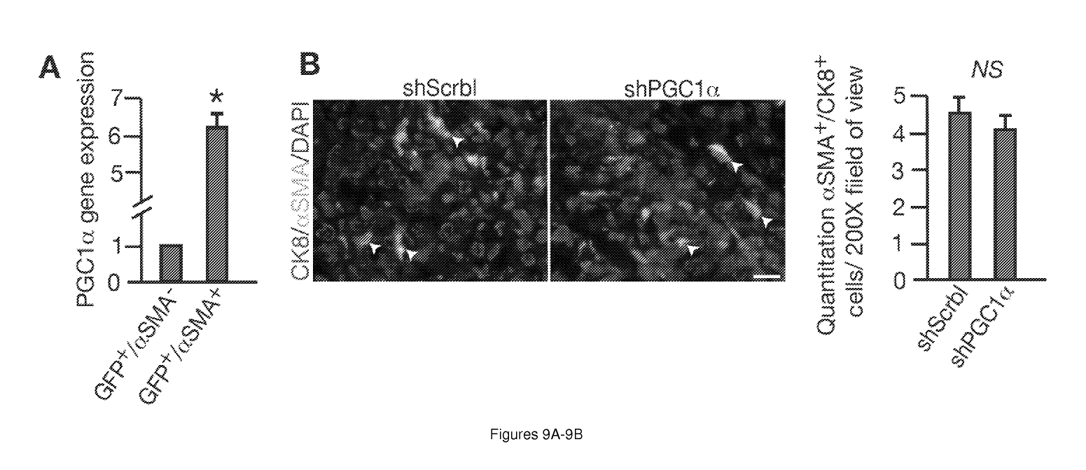

Our studies pointed to the possibility that PGC1α expression is essential for intravasation of the cancer cells into the circulation. Therefore, we next probed whether extravasation of cancer cells is also similarly impaired when PGC1α is suppressed. We monitored lung colonization and lung metastatic nodule formation in mice following intravenous injection of 4T1shPGC1α and control 4T1shScrb1 cells. Our results indicated that metastatic lung colonization and nodule formation was significantly impaired with suppressed PGC1α expression (FIG. 8A-C). Similar results were also observed in mice injected intravenously with MDA-MB-231shPGC1α and MD-MB-231shScrb1 cells and (FIG. 8D-F), B16F10shPGC1α and B16F10shScrb1 cells (FIG. 8G-I). Taken together, our results support an important role for PGC1α-mediated mitochondrial biogenesis and oxidative phosphorylation in facilitating migration, invasion, and extravasation/intravasation of cancer cells.

### Motility of Cancer Cells is Functional Fueled by Mitochondrial Respiration

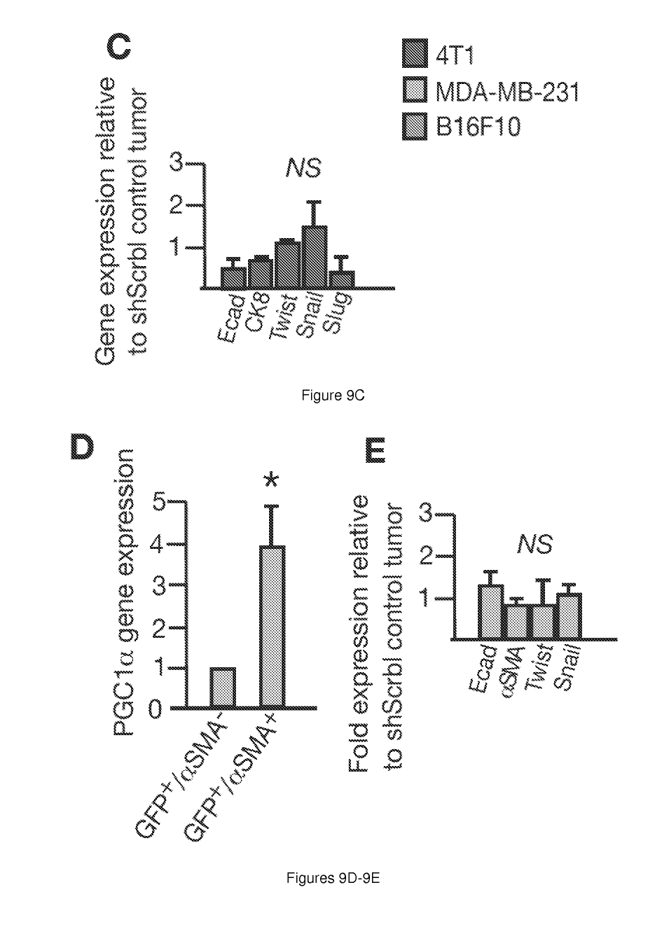

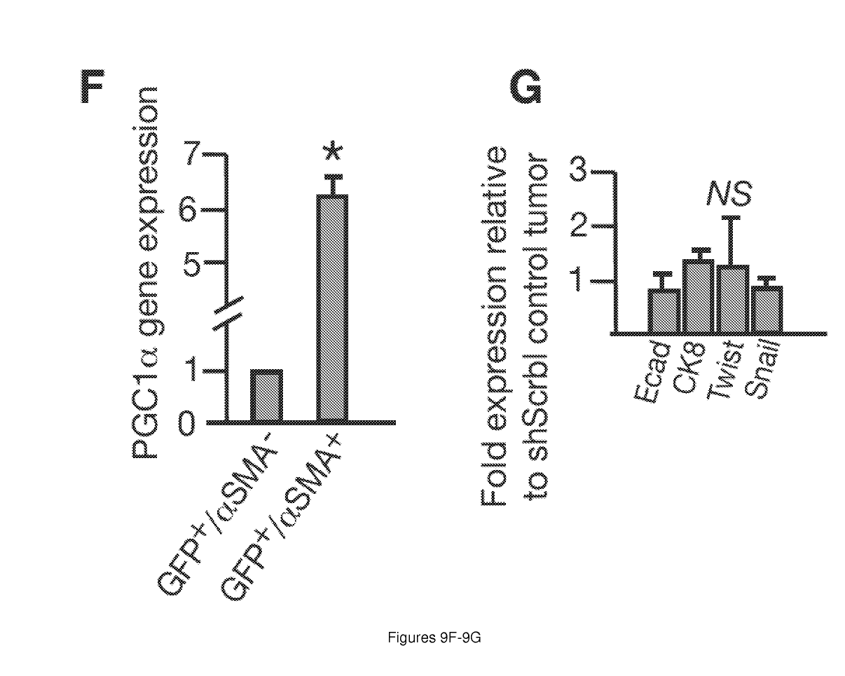

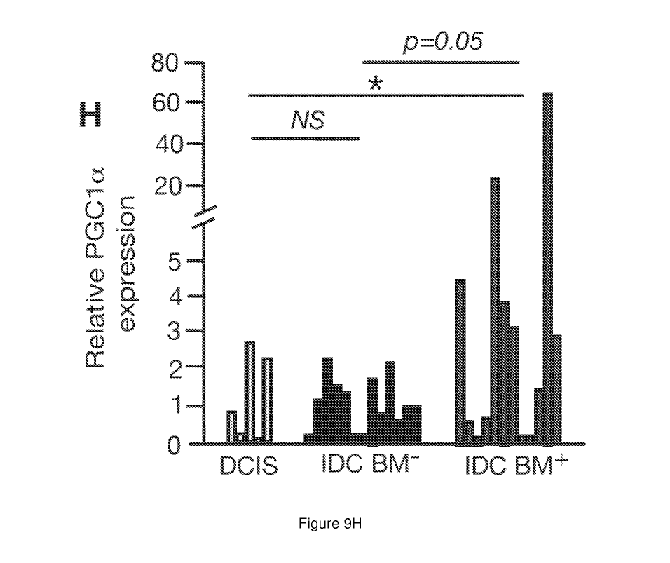

GFP⁺4T1-BCC from the primary tumors were labeled for the mesenchymal marker, αSMA, and subsequently FACS purified based on GFP and αSMA double labeling. Cancer cells exhibiting an EMT program (GFP⁺/αSMA⁺) express significantly higher levels of PGC1α when compared to cancer cells without EMT program (GFP⁺/αSMA⁻) (FIG. 9A). We next evaluated whether tumors with suppressed PGC1α expression have impaired migratory and EMT gene expression profile. Double immunolabeling for CK8 (epithelial marker) and αSMA (mesenchymal marker) revealed a similar number of double positive cancer cells in 4T1shPGC1α and control 4T1shScrb1 primary tumors, suggesting an equal frequency of cancer cells acquiring EMT program (FIG. 9B). Q-PCR analyses for mesenchymal and epithelial genes also revealed comparable induction of EMT program in both 4T1shPGC1α and control 4T1shScrb1 primary tumors (FIG. 9C). These results were consistent with the observation that suppression of PGC1α did not impact the expression of EMT related genes (FIGS. 3J, 4J and 5J).

Similar findings were observed in MDA-MB-231 and B16F10 tumors: while PGC1α expression was significantly induced in MDA-MB-231-GFP⁺/αSMA⁺ (FIG. 9D) and B16F10-GFP⁺/αSMA⁺ (FIG. 9F) cells purified from the primary tumors, the frequency of EMT remained unaffected by the suppression of PGC1α expression (FIG. 9E, G). Collectively these findings suggest that while modulating mitochondrial respiration in cancer cells via PGC1α expression does not impact their ability to acquire a gene expression signature characteristic of EMT, it functionally impairs their movement.

Enhanced PGC1α Expression is Associated with Invasive Breast Cancer with Bone Micrometastasis

Microdissected neoplastic cells from breast tumors resected from patients diagnosed with ductal carcinoma in situ (DCIS, n=5) and invasive ductal carcinomas (IDC), categorized based on bone marrow micrometastasis positivity (BM⁺ (n=12) vs. BM⁻ (n=13)) were assessed for PGC1α gene expression. PGC1α expression was upregulated in several BM⁺ IDC patients, when compared to DCIS and BM⁻ IDC patients (FIG. 9H). Not all BM⁺ IDC patients showed significant increase in PGC1α expression, possibly reflecting heterogeneity across collected samples in their relative content of cancer cells that have acquired a migratory phenotype. Nevertheless, this preliminary clinical study offers insight into possible association of PGC1α expression with invasive cancer.

### Other Embodiments

All publications, patent applications, and patents mentioned in this specification are herein incorporated by reference.

Various modifications and variations of the described method and system of the invention will be apparent to those skilled in the art without departing from the scope and spirit of the invention. Although the invention has been described in connection with specific desired embodiments, it should be understood that the invention as claimed should not be unduly limited to such specific embodiments. Indeed, various modifications of the described modes for carrying out the invention that are obvious to those skilled in the fields of medicine, pharmacology, or related fields are intended to be within the scope of the invention.

What is claimed is:

1. A method of treating a subject having a metastatic disease, said method comprising administering to said subject an antagonist of mitochondrial respiration, in an amount sufficient to treat said metastatic disease.

2. The method of claim 1, further comprising:
a) determining the level of mitochondrial respiration in a sample from said subject, and
b) administering to a subject having increased levels of mitochondrial respiration an antagonist that inhibits mitochondrial respiration in an amount sufficient to treat said metastatic disease.

3. The method of claim 2, wherein the levels of mitochondrial respiration is determined based on increased PGC1α activity.

4. The method of claim 2, wherein said sample comprises cancer cells.

5. The method of claim 2, wherein said cancer cells are circulating tumor cells.

6. The method of claim 1, wherein said antagonist is an RNAi agent, a small molecule inhibitor, or an antibody.

7. The method of claim 1, wherein said antagonist is a PGC1α antagonist.

8. The method of claim 1, wherein said PGC1α antagonist is an RNAi agent, or an anti-PGC1α antibody.

9. The method of claim 1, wherein said antagonist is administered with an anticancer agent.

10. The method of claim 1, wherein said metastatic disease is selected from the group consisting of: leukemia, brain cancer, bladder cancer, breast cancer, cervical cancer, colorectal cancer, endometrial cancer, esophageal cancer, head and neck cancer, liver cancer, lung cancer, lymphoma, ovarian cancer, pancreatic cancer, prostate cancer, renal cancer, skin cancer, stomach cancer, testis cancer, thyroid cancer, and urothelial cancer.

11. The method of claim 1, wherein said metastatic disease is breast cancer and said breast cancer is selected from the group consisting of: ductal carcinoma, invasive ductal carcinoma, tubular carcinoma, medullary carcinoma, mucinous carcinoma, papillary carcinoma, cribriform carcinoma, invasive lobular carcinoma, inflammatory breast cancer, lobular carcinoma, male breast cancer, Paget's Disease, and phyllodes tumors.

12. A method for diagnosing a subject as having, or having a predisposition to a metastatic disease, said method comprising:
a) determining the level of mitochondrial respiration in a sample from said subject,
b) comparing said level of mitochondrial respiration with a normal reference sample, wherein the presence of an increased level of mitochondrial respiration, as compared to said normal reference sample, results in diagnosing said subject as having, or having a predisposition to said metastatic disease, and
c) administering to said subject an antagonist that inhibits mitochondrial respiration, in an amount sufficient to treat said metastatic disease.

13. The method of claim 12, wherein said level of mitochondrial respiration is determined based on increased PGC1α activity.

14. The method of claim 12, wherein said sample comprises cancer cells.

15. The method of claim 12, wherein said cancer cells are circulating tumor cells.

16. The method of claim 12, wherein said antagonist is an RNAi agent, a small molecule inhibitor, or an antibody.

17. The method of claim 12, wherein said antagonist is a PGC1α antagonist.

18. The method of claim 12, wherein said metastatic disease is selected from the group consisting of: leukemia, brain cancer, bladder cancer, breast cancer, cervical cancer, colorectal cancer, endometrial cancer, esophageal cancer, head and neck cancer, liver cancer, lung cancer, lymphoma, ovarian cancer, pancreatic cancer, prostate cancer, renal cancer, skin cancer, stomach cancer, testis cancer, thyroid cancer, and urothelial cancer.

19. The method of claim 12, wherein said metastatic disease is breast cancer and said breast cancer is selected from the group consisting of: ductal carcinoma, invasive ductal carcinoma, tubular carcinoma, medullary carcinoma, mucinous carcinoma, papillary carcinoma, cribriform carcinoma, invasive lobular carcinoma, inflammatory breast cancer, lobular carcinoma, male breast cancer, Paget's Disease, and phyllodes tumors.


SEQUENCE LISTING (64 sequences)

SEQ ID NO: 1 - DNA, 20 bp, Artificial Sequence
  Description of Artificial Sequence Synthetic primer
ggctgtattcccctccatcg

SEQ ID NO: 2 - DNA, 22 bp, Artificial Sequence
  Description of Artificial Sequence Synthetic primer
ccagttggtaacaatgccatgt

SEQ ID NO: 3 - DNA, 22 bp, Artificial Sequence
  Description of Artificial Sequence Synthetic primer
agccgtgaccactgacaacgag

SEQ ID NO: 4 - DNA, 22 bp, Artificial Sequence
  Description of Artificial Sequence Synthetic primer
gctcatggttctgagtgctaag

SEQ ID NO: 5 - DNA, 23 bp, Artificial Sequence
  Description of Artificial Sequence Synthetic primer
ctgctgtctctttcggatagatc

SEQ ID NO: 6 - DNA, 19 bp, Artificial Sequence
  Description of Artificial Sequence Synthetic primer
cggaaacggcctcatctct

SEQ ID NO: 7 - DNA, 21 bp, Artificial Sequence
  Description of Artificial Sequence Synthetic primer
gaggtgtggcagtgttcsttg

SEQ ID NO: 8 - DNA, 19 bp, Artificial Sequence
  Description of Artificial Sequence Synthetic primer
ggctgcattgtgaccttca

SEQ ID NO: 9 - DNA, 16 bp, Artificial Sequence
  Description of Artificial Sequence Synthetic primer
aggaagccccgatgga

SEQ ID NO: 10 - DNA, 20 bp, Artificial Sequence
  Description of Artificial Sequence Synthetic primer
gagaggcctgggatgctctt

SEQ ID NO: 11 - DNA, 21 bp, Artificial Sequence
  Description of Artificial Sequence Synthetic primer
aagcagttcaacgagaacgaa

SEQ ID NO: 12 - DNA, 20 bp, Artificial Sequence
  Description of Artificial Sequence Synthetic primer
cgtacagcgcagaaaacagg

SEQ ID NO: 13 - DNA, 22 bp, Artificial Sequence
  Description of Artificial Sequence Synthetic primer
ggaagaccctaatctagtcccg

SEQ ID NO: 14 - DNA, 22 bp, Artificial Sequence
  Description of Artificial Sequence Synthetic primer
ccactattctcttgttgctgat

SEQ ID NO: 15 - DNA, 23 bp, Artificial Sequence
  Description of Artificial Sequence Synthetic primer
attggcaagagagccatttctac

SEQ ID NO: 16 - DNA, 21 bp, Artificial Sequence
  Description of Artificial Sequence Synthetic primer
caacactcccatgtgctcgaa

SEQ ID NO: 17 - DNA, 19 bp, Artificial Sequence
  Description of Artificial Sequence Synthetic primer
gagactgggcgtgtgttag

SEQ ID NO: 18 - DNA, 21 bp, Artificial Sequence
  Description of Artificial Sequence Synthetic primer
ctcgacgcaataccatcacca

SEQ ID NO: 19 - DNA, 21 bp, Artificial Sequence
  Description of Artificial Sequence Synthetic primer
aaagggaggcaagcataagac

SEQ ID NO: 20 - DNA, 21 bp, Artificial Sequence
  Description of Artificial Sequence Synthetic primer
gaacagaccgtggagatttgg

SEQ ID NO: 21 - DNA, 21 bp, Artificial Sequence
  Description of Artificial Sequence Synthetic primer
atgggcggaatggtctctttc

SEQ ID NO: 22 - DNA, 21 bp, Artificial Sequence
  Description of Artificial Sequence Synthetic primer
tggggaccttgtcttcatcat

SEQ ID NO: 23 - DNA, 21 bp, Artificial Sequence
  Description of Artificial Sequence Synthetic primer
aggtggtgatagccggtatgt

SEQ ID NO: 24 - DNA, 21 bp, Artificial Sequence
  Description of Artificial Sequence Synthetic primer
tgggtaatccatagagcccag

SEQ ID NO: 25 - DNA, 21 bp, Artificial Sequence
  Description of Artificial Sequence Synthetic primer
tccatcagggtgactcagaaa

SEQ ID NO: 26 - DNA, 19 bp, Artificial Sequence
  Description of Artificial Sequence Synthetic primer
ccagcttcaaggggctcaa

SEQ ID NO: 27 - DNA, 18 bp, Artificial Sequence
  Description of Artificial Sequence Synthetic primer
cgggagtccgcagtctta

SEQ ID NO: 28 - DNA, 20 bp, Artificial Sequence
  Description of Artificial Sequence Synthetic primer
tgaatcttgctcagcttgtc

SEQ ID NO: 29 - DNA, 24 bp, Artificial Sequence
  Description of Artificial Sequence Synthetic primer
tccaaacccactcggatgtgaaga

SEQ ID NO: 30 - DNA, 24 bp, Artificial Sequence
  Description of Artificial Sequence Synthetic primer
ttggtgcttgtggagcaaggacat

SEQ ID NO: 31 - DNA, 20 bp, Artificial Sequence
  Description of Artificial Sequence Synthetic primer
ggcaccactgaaccctaagg

SEQ ID NO: 32 - DNA, 23 bp, Artificial Sequence
  Description of Artificial Sequence Synthetic primer
acaataccagttgtacgtccaga

SEQ ID NO: 33 - DNA, 20 bp, Artificial Sequence
  Description of Artificial Sequence Synthetic primer
gagcctgagtcctgcagtcc

SEQ ID NO: 34 - DNA, 20 bp, Artificial Sequence
  Description of Artificial Sequence Synthetic primer
tgtattgctgcttggcctca

SEQ ID NO: 35 - DNA, 24 bp, Artificial Sequence
  Description of Artificial Sequence Synthetic primer
cacattcgaacccacacattgcct

SEQ ID NO: 36 - DNA, 24 bp, Artificial Sequence
  Description of Artificial Sequence Synthetic primer
tgtgccctcaggtttgatctgtct

SEQ ID NO: 37 - DNA, 21 bp, Artificial Sequence
  Description of Artificial Sequence Synthetic primer
aggagaagtctgccgttactg

SEQ ID NO: 38 - DNA, 21 bp, Artificial Sequence
  Description of Artificial Sequence Synthetic primer
cttcatccacgttcaccttgc

SEQ ID NO: 39 - DNA, 20 bp, Artificial Sequence
  Description of Artificial Sequence Synthetic primer
gctttctgggtggactcaac

SEQ ID NO: 40 - DNA, 20 bp, Artificial Sequence
  Description of Artificial Sequence Synthetic primer
ctgctagcaagtttgcctca

SEQ ID NO: 41 - DNA, 20 bp, Artificial Sequence
  Description of Artificial Sequence Synthetic primer
aggaacacggagtgacccaa

SEQ ID NO: 42 - DNA, 21 bp, Artificial Sequence
  Description of Artificial Sequence Synthetic primer
tgcatgtgcttctatggtagc

SEQ ID NO: 43 - DNA, 21 bp, Artificial Sequence
  Description of Artificial Sequence Synthetic primer
atggcttcaaggttacttcgc

SEQ ID NO: 44 - DNA, 20 bp, Artificial Sequence
  Description of Artificial Sequence Synthetic primer
ccctttggggccagtacatt

SEQ ID NO: 45 - DNA, 20 bp, Artificial Sequence
  Description of Artificial Sequence Synthetic primer
actaccccatgccagaagag

SEQ ID NO: 46 - DNA, 21 bp, Artificial Sequence
  Description of Artificial Sequence Synthetic primer
tcattggagcgacggttcatc

SEQ ID NO: 47 - DNA, 21 bp, Artificial Sequence
  Description of Artificial Sequence Synthetic primer
tgcaaggaacttccatgcctc

SEQ ID NO: 48 - DNA, 22 bp, Artificial Sequence
  Description of Artificial Sequence Synthetic primer
cgcccagtttcttcaagatcaa

SEQ ID NO: 49 - DNA, 20 bp, Artificial Sequence
  Description of Artificial Sequence Synthetic primer
ctttgggcggaagacaggtc

SEQ ID NO: 50 - DNA, 21 bp, Artificial Sequence
  Description of Artificial Sequence Synthetic primer
ttattggcggctgtgtaagag

SEQ ID NO: 51 - DNA, 23 bp, Artificial Sequence
  Description of Artificial Sequence Synthetic primer
tgagactagccaaacaatctcgt

SEQ ID NO: 52 - DNA, 22 bp, Artificial Sequence
  Description of Artificial Sequence Synthetic primer
agaaagtagaagctccgatcct

SEQ ID NO: 53 - DNA, 23 bp, Artificial Sequence
  Description of Artificial Sequence Synthetic primer
aaggacctgtctaggtttgatgc

SEQ ID NO: 54 - DNA, 21 bp, Artificial Sequence
  Description of Artificial Sequence Synthetic primer
tggcttcataggtgacttcca

SEQ ID NO: 55 - DNA, 21 bp, Artificial Sequence
  Description of Artificial Sequence Synthetic primer
agcagtcagtttgtgaccagg

SEQ ID NO: 56 - DNA, 21 bp, Artificial Sequence
  Description of Artificial Sequence Synthetic primer
atctcctagttcgggtgcttt

SEQ ID NO: 57 - DNA, 20 bp, Artificial Sequence
  Description of Artificial Sequence Synthetic primer
cgagagctacacgttcacgg

SEQ ID NO: 58 - DNA, 21 bp, Artificial Sequence
  Description of Artificial Sequence Synthetic primer
gggtgtcgagggaaaaatagg

SEQ ID NO: 59 - DNA, 18 bp, Artificial Sequence
  Description of Artificial Sequence Synthetic primer
cgggagtccgcagtctta

SEQ ID NO: 60 - DNA, 20 bp, Artificial Sequence
  Description of Artificial Sequence Synthetic primer
tgaatcttgctcagcttgtc

SEQ ID NO: 61 - DNA, 18 bp, Artificial Sequence
  Description of Artificial Sequence Synthetic primer
gaggcggtggcagactag

SEQ ID NO: 62 - DNA, 19 bp, Artificial Sequence
  Description of Artificial Sequence Synthetic primer
gacacatcggtcagaccag

SEQ ID NO: 63 - DNA, 20 bp, Artificial Sequence
  Description of Artificial Sequence Synthetic primer
gctttcagcttccctgaaca

SEQ ID NO: 64 - DNA, 20 bp, Artificial Sequence
  Description of Artificial Sequence Synthetic primer
ggagctgcttcacaggattc